(19) United States
(12) Patent Application Publication
(10) Pub. No.: US 2013/0089577 A1
(43) Pub. Date: Apr. 11, 2013

(54) NOVEL ESTER CONTAINING COMPOSITIONS AND METHODS

(71) Applicants: Joseph P. St. Laurent, Lakeville, MA (US); Scott A. Goodrich, Stoughton, MA (US); Gerald S. Jones, JR., Norwood, MA (US)

(72) Inventors: Joseph P. St. Laurent, Lakeville, MA (US); Scott A. Goodrich, Stoughton, MA (US); Gerald S. Jones, JR., Norwood, MA (US)

(21) Appl. No.: 13/634,166

(22) PCT Filed: Mar. 11, 2011
(86) PCT No.: PCT/US11/28200

§ 371 Date: Dec. 21, 2012

Related U.S. Application Data
(60) Provisional application No. 61/313,100, filed on Mar. 11, 2010.
(60) Provisional application No. 61/355,664, filed on Jun. 17, 2010.
(60) Provisional application No. 61/372,371, filed on Aug. 10, 2010.

(51) Int. Cl. A61K 47/14; A61K 31/436; A61K 31/58; A61K 31/415; A61K 31/167; A61K 31/192

(52) U.S. Cl. CPC A61K 47/14; A61K 31/167; A61K 31/192; A61K 31/58; A61K 31/415; A61K 31/436
USPC 424/400; 514/785; 514/626; 514/570; 514/174; 514/406; 514/291

(57) ABSTRACT

Described herein are compositions (e.g., a pharmaceutical composition) and methods for controlling the delivery of a therapeutic agent, and their use in the treatment and/or prevention of diseases and disorders.

### CLAIM OF PRIORITY

This application claims priority to U.S. Ser. No. 61/313,100, filed Mar. 11, 2010, U.S. Ser. No. 61/355,664, filed Jun. 17, 2010 and U.S. Ser. No. 61/372,371, filed Aug. 10, 2010, the contents of each of which is incorporated by reference in its entirety.

### BACKGROUND OF THE INVENTION

Delivery of a therapeutic agent through the skin of a subject can provide for an advantageous form of systemic or local delivery. Topical delivery allows a therapeutic agent to by-pass the digestive track and avoid first pass metabolism. The ability to control the permeation rate and depth of penetration of a therapeutic agent would allow tremendous benefits over current drug delivery technologies.

### SUMMARY OF INVENTION

The inventors have discovered novel ways of controlling the delivery of a therapeutic agent through the skin of a subject. For example, the inventors have discovered that the use of a plurality of esters, e.g., alpha hydroxy carboxylic acid esters or a combination of an alpha hydroxy carboxylic acid ester with an additional ester such as an octisalate ester, are effective in delivery of a therapeutic agent to a subject through the skin or mucous membrane of the subject. Moreover, the inventors have discovered that varying the relative amount of the esters can result in a change in the relative rate of delivery of a therapeutic agent through the skin of a subject. For example, if two esters are used in combination with a therapeutic agent, the inventors have discovered that changing the relative amount of the first ester to the second ester can result in a change in the relative rate of delivery of the therapeutic agent to the subject. By way of further example, a relative ratio of 1:1 of a first ester to a second ester for delivery of therapeutic agent A will give a different rate of delivery of therapeutic agent A than formulation of therapeutic agent A with a relative ratio of 4:1 of the first ester to the second ester. It is the manipulation of the relative amounts of these esters that can be used to “dial-in” specific rates of delivery of a therapeutic agent. This concept can also be expanded to include more than two esters, e.g., 2, 3, 4, 5, 6, 7, 8, 9, 10 or more. Applicants' ability to provide for each ester in ultra pure form provides a unique ability to “dial-in” a delivery rate with specific control, due to Applicants' ability to maintain tight control over the components in each formulation. Accordingly, in one aspect, the present invention is directed to topical delivery of drugs via the dialed-In Delivery© technology platform which is associated with the compositions and methods described herein.

In another aspect, the invention is directed to a composition of esters, the composition comprising an α-hydroxy carboxylic acid ester and a second ester, wherein the α-hydroxy carboxylic acid ester and the second ester are not the same; and wherein less than 10% (e.g., by weight or volume) of the composition comprises fatty alcohols and/or an alkyl lactyllactate and/or other related substances.

In certain embodiments, the composition is a pharmaceutical composition. In some embodiments, the composition further comprises a drug (e.g., a small molecule drug, a peptide or polypeptide, a protein or a hormone).

In some embodiments, the drug is an analgesic (e.g., ibuprofen).

In some embodiments, the drug is an antiarrhythmic or an anesthetic (e.g., a local anesthetic such as lidocaine).

In some embodiments, the drug is selected from the group consisting of aclometasone, acyclovir, alitretinoin, alprostadil, amcinonide, amlexanox, anthralin, azelaic acid, bacitracin, becaplermin, betamethasone, betamethasone valerate, benzocaine, benzoyl peroxide, budenoside, bupivacaine, buprenorphine, bupropion, butenafine, butoconazole, calcipotriene, calcitriol, capsaicin, celecoxib, chlorhexidene gluconate, ciclopirox, clindamycin, clobetasol propionate, clocortilone, clotrimazole, crotamiton, dehydroergotamine, desonide dipropionate, desoximetasone, desoximetasone pivalate, disulfuram, diazepam, dibucaine, diclofenac, diflorasone diacetate, diflunisal, diphenhydramine, donepezil, doxepin, erythromycin, famciclovir, fentanyl, fluandrenolide, flucinolone acetonide, flucinonide, flunisolide, fluorouracil, fluoxetine, fluticasone, fluticasone propionate, fluvoxamine, glycolic acid, halcinonide, halobetasol propionate, hydrocodone, hydrocortisone, hydrocortisone valerate, hydroxyzine, ibuprofen, imiquimod, indomethacin, isosorbide dintrate, ketoconazole, ketoprofen, ketorolac, lidocaine, lindane, mafenide acetate, meperidine, metoclopramide, metronidazole, miconazole, mometasone, mometasone furoate, morphine, mupirocin, naftifine, naloxone, naltrexone, naproxen, neomycin, nitroglycerin, nystatin, ondansetron, oxiconazole, oxycodone, paroxetine, penciclovir, permethrin, phenylephrine, piperonyl butoxide, piroxicam, podofilox, polymyxin, pramoxine, prilocalne, prochlorperazine, promethazine, pyrethrins, rofecoxib, scopolamine, sertraline, sildenafil, sufentanil, sulindac, sumatriptan, tacrine, tacrolimus, tadalafil, tazarotene, terbinafine, terconazole, testosterone, tioconazole, tolnaftate, trazodone, tretinoin, tramcinolone, triazolam, valacyclovir, vardenafil, varenicline and zolpidem.

In certain embodiments, the second ester is a compound of formula (I):

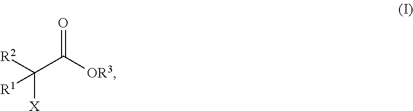

wherein
each R¹ is independently selected from the group consisting of: H, C₁-C₂₀ straight chained or branched alkyl, C₃-C₁₀ cycloalkyl, substituted C₁-C₂₀ alkyl, arylalkyl, aryl, substituted aryl and heteroaryl;
each R² is independently selected from the group consisting of: H and C₁-C₂₀ alkyl;
R³ is a C₁₂-C₄₀ alkyl; and

### X is NR¹R¹ or OR¹;

In some embodiments, the α-hydroxy carboxylic acid ester is a compound of formula (II):

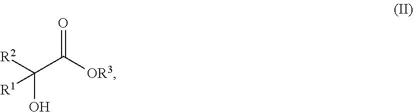

wherein R¹, R² and R³ are as defined in formula (I).

In some embodiments, the second ester is a compound of formula (II):

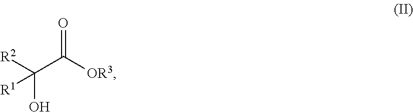

wherein R¹, R² and R³ are as defined in formula (I).

In some embodiments, the α-hydroxy carboxylic acid ester and the second esters are compounds of formula (II):

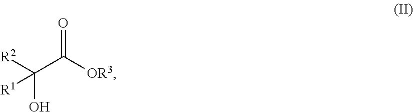

wherein R¹, R² and R³ are as described in formula (I).

In certain embodiments, the composition further comprises a third ester. In some embodiments, the composition further comprises a fourth ester. In some embodiments, the composition further comprises a fifth ester. In some embodiments, the composition comprises more than five or more esters.

In certain embodiments, the α-hydroxy carboxylic acid ester, the second ester and the third ester are not the same. In some embodiments, the α-hydroxy carboxylic acid ester, the second ester, the third ester and the fourth ester are not the same. In some embodiments, the second ester, the third ester, the fourth ester and the fifth are not the same.

In certain embodiments, the α-hydroxy carboxylic acid ester and the second ester used in the composition are selected from the group consisting of lauryl lactate, lauryl mandelate, myristyl lactate, cetyl lactate, stearyl lactate, ethyl hexyl lactate and dimethyl octyl lactate. In some embodiments, the α-hydroxy carboxylic acid ester, the second ester and the third ester used in the composition are selected from the group consisting of lauryl lactate, myristyl lactate, cetyl lactate, stearyl lactate, ethyl hexyl lactate and dimethyl octyl lactate. In some embodiments, the α-hydroxy carboxylic acid ester, the second ester, the third ester and the fourth ester used in the composition are selected from the group consisting of lauryl lactate, myristyl lactate, cetyl lactate, stearyl lactate, ethyl hexyl lactate and dimethyl octyl lactate. In some embodiments, the α-hydroxy carboxylic acid ester, the second ester, the third ester, the fourth ester and the fifth ester used in the composition are selected from the group consisting of lauryl lactate, myristyl lactate, cetyl lactate, stearyl lactate, ethyl hexyl lactate and dimethyl octyl lactate. In some embodiments, the esters are chosen in ratios so as to provide a preselected delivery of a drug through the skin or mucousal membrane.

In certain embodiments, the composition comprises less than 5% (e.g., by weight or volume) fatty alcohols and/or alkyl lactyllactate and/or other related substances. In certain embodiments, the composition comprises less than 3% (e.g., by weight or volume) fatty alcohols and/or alkyl lactyllactate. In certain embodiments, the composition comprises less than 1% (e.g., by weight or volume) fatty alcohols and/or alkyl lactyllactate. In some embodiments, the composition is substantially free of fatty alcohols and/or alkyl lactyllactate.

In certain embodiments, the α-hydroxy carboxylic acid ester is present in a racemic mixture (e.g., less than 10% enantiomeric excess of either the R or S stereoisomer). In some embodiments, the α-hydroxy carboxylic acid ester is present in an enantiomeric excess of the R stereoisomer (e.g., 10%, 50%, 75%, 85%, 90%, 95%, 97% or greater). In some embodiments, the α-hydroxy carboxylic acid ester is present in an enantiomeric excess of the S stereoisomer (e.g., 10%, 50%, 75%, 85%, 90%, 95%, 97% or greater).

In certain embodiments, the second ester is present in a racemic mixture (e.g., less than 10% enantiomeric excess of either the R or S stereoisomer). In some embodiments, the second ester is present in an enantiomeric excess of the R stereoisomer (e.g., 10%, 50%, 75%, 85%, 90%, 95%, 97% or greater). In some embodiments, the second ester is present in an enantiomeric excess of the S stereoisomer (e.g., 10%, 50%, 75%, 85%, 90%, 95%, 97% or greater).

In certain embodiments, the third ester is present in a racemic mixture (e.g., less than 10% enantiomeric excess of either the R or S stereoisomer). In some embodiments, the third ester is present in an enantiomeric excess of the R stereoisomer (e.g., 10%, 50%, 75%, 85%, 90%, 95%, 97% or greater). In some embodiments, the third ester is present in an enantiomeric excess of the S stereoisomer (e.g., 10%, 50%, 75%, 85%, 90%, 95%, 97% or greater).

In certain embodiments, the fourth ester is present in a racemic mixture (e.g., less than 10% enantiomeric excess of either the R or S stereoisomer). In some embodiments, the fourth ester is present in an enantiomeric excess of the R stereoisomer (e.g., 10%, 50%, 75%, 85%, 90%, 95%, 97% or greater). In some embodiments, the fourth ester is present in an enantiomeric excess of the S stereoisomer (e.g., 10%, 50%, 75%, 85%, 90%, 95%, 97% or greater).

In certain embodiments, the fifth ester is present in a racemic mixture (e.g., less than 10% enantiomeric excess of either the R or S stereoisomer). In some embodiments, the fifth ester is present in an enantiomeric excess of the R stereoisomer (e.g., 10%, 50%, 75%, 85%, 90%, 95%, 97% or greater). In some embodiments, the fifth ester is present in an enantiomeric excess of the S stereoisomer (e.g., 10%, 50%, 75%, 85%, 90%, 95%, 97% or greater).

In certain embodiments, the composition is configured for topical administration. In some embodiments, the composition is configured for administration in the form of a patch. In some embodiments, the composition is in the form of an oil-in-water emulsion. In some embodiments, the composition is in the form of a water-in-oil emulsion. In some embodiments, the composition is in the form of a thickened aqueous gel. In some embodiments, the composition is in the form of a hydrophilic gel. In some embodiments, the composition is in the form of an ahydrous gel. In some embodiments, the composition is in the form of a solution. In some embodiments, the composition is in the form of a hydrophobic gel.

In certain embodiments, the composition consists essentially of an α-hydroxy carboxylic acid ester and a second ester, wherein the esters are not the same. In some embodiments, the composition consists of an α-hydroxy carboxylic acid ester and a second ester, wherein the esters are not the same.

In certain embodiments, ratio of esters are chosen so as to provide a preselected rate of a drug through the skin or mucosal membrane of a subject. The drugs can also be selected to provide for a preselected rate of a delivery through the skin or a mucousal membrane. Exemplary ratios may include wherein the α-hydroxy carboxylic acid ester and second ester are present in a range of 99:1 to 1:99 (e.g, 10:1, 5:1, 2:1, 1:1, 1:2, 1:5, 1:10 or 1:99).

In another aspect, the invention is directed to a composition of esters, the composition comprising an α-hydroxy carboxylic acid ester and a second ester, wherein the α-hydroxy carboxylic acid ester and the second ester are not the same; and wherein at least 90% (e.g., by weight or volume) of the composition is comprised of esters.

In certain embodiments, the composition is a pharmaceutical composition. In some embodiments, the composition further comprises a drug (e.g., a small molecule drug, a peptide or polypeptide, a protein or a hormone).

In certain embodiments, at least 90% (e.g., by weight or volume) of the composition is comprised of the α-hydroxy carboxylic acid ester and the second ester.

In certain embodiments, the second ester is a compound of formula (I):

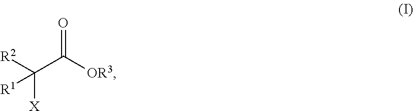

wherein
each R¹ is independently selected from the group consisting of: H, C₁-C₂₀ straight chained or branched alkyl, C₃-C₁₀ cycloalkyl, substituted C₁-C₂₀ alkyl, arylalkyl, aryl, substituted aryl and heteroaryl;
each R² is independently selected from the group consisting of: H and C₁-C₂₀ alkyl;
R³ is a C₁₂-C₄₀ alkyl; and

### X is NR¹R¹ or OR¹;

In some embodiments, the α-hydroxy carboxylic acid ester is a compound of formula (II):

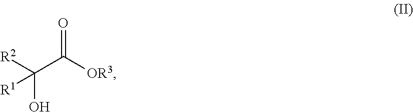

wherein R¹, R² and R³ are as defined in formula (I).

In some embodiments, the second ester is a compound of formula (II):

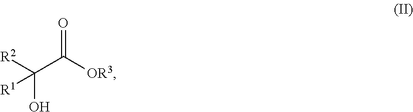

wherein R¹, R² and R³ are as defined in formula (I).

In some embodiments, the α-hydroxy carboxylic acid ester and the second esters are compounds of formula (II):

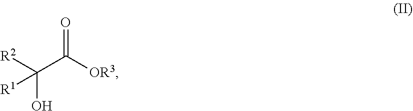

wherein R¹, R² and R³ are as described in formula (I).

In certain embodiments, the composition is substantially free of fatty alcohols and/or alkyl lactyllactates and/or other related substances.

In certain embodiments, the composition further comprises a third ester. In some embodiments, the composition further comprises a fourth ester. In some embodiments, the composition further comprises a fifth ester. In some embodiments, the composition comprises more than five or more esters.

In certain embodiments, the α-hydroxy carboxylic acid ester, the second ester and the third ester are not the same. In some embodiments, the α-hydroxy carboxylic acid ester, the second ester, the third ester and the fourth ester are not the same. In some embodiments, the α-hydroxy carboxylic acid ester, the second ester, the third ester, the fourth ester and the fifth are not the same.

In certain embodiments, the α-hydroxy carboxylic acid ester is present in a racemic mixture (e.g., less than 10% enantiomeric excess of either the R or S stereoisomer). In some embodiments, the α-hydroxy carboxylic acid ester is present in an enantiomeric excess of the R stereoisomer (e.g., 10%, 50%, 75%, 85%, 90%, 95%, 97% or greater). In some embodiments, the α-hydroxy carboxylic acid ester is present in an enantiomeric excess of the S stereoisomer (e.g., 10%, 50%, 75%, 85%, 90%, 95%, 97% or greater).

In certain embodiments, the second ester is present in a racemic mixture (e.g., less than 10% enantiomeric excess of either the R or S stereoisomer). In some embodiments, the second ester is present in an enantiomeric excess of the R stereoisomer (e.g., 10%, 50%, 75%, 85%, 90%, 95%, 97% or greater). In some embodiments, the second ester is present in an enantiomeric excess of the S stereoisomer (e.g., 10%, 50%, 75%, 85%, 90%, 95%, 97% or greater).

In certain embodiments, the third ester is present in a racemic mixture (e.g., less than 10% enantiomeric excess of either the R or S stereoisomer). In some embodiments, the third ester is present in an enantiomeric excess of the R stereoisomer (e.g., 10%, 50%, 75%, 85%, 90%, 95%, 97% or greater). In some embodiments, the third ester is present in an enantiomeric excess of the S stereoisomer (e.g., 10%, 50%, 75%, 85%, 90%, 95%, 97% or greater).

In certain embodiments, the fourth ester is present in a racemic mixture (e.g., less than 10% enantiomeric excess of either the R or S stereoisomer). In some embodiments, the fourth ester is present in an enantiomeric excess of the R stereoisomer (e.g., 10%, 50%, 75%, 85%, 90%, 95%, 97% or greater). In some embodiments, the fourth ester is present in an enantiomeric excess of the S stereoisomer (e.g., 10%, 50%, 75%, 85%, 90%, 95%, 97% or greater).

In certain embodiments, the fifth ester is present in a racemic mixture (e.g., less than 10% enantiomeric excess of either the R or S stereoisomer). In some embodiments, the fifth ester is present in an enantiomeric excess of the R stereoisomer (e.g., 10%, 50%, 75%, 85%, 90%, 95%, 97% or greater). In some embodiments, the fifth ester is present in an enantiomeric excess of the S stereoisomer (e.g., 10%, 50%, 75%, 85%, 90%, 95%, 97% or greater).

In certain embodiments, the composition is configured for topical administration. In some embodiments, the composition is configured for administration in the form of a patch. In some embodiments, the composition is in the form of an oil-in-water emulsion. In some embodiments, the composition is in the form of a water-in-oil emulsion. In some embodiments, the composition is in the form of a thickened aqueous gel. In some embodiments, the composition is in the form of a hydrophilic gel. In some embodiments, the composition is in the form of a hydrophobic gel. In some embodiments, the composition is in the form of an anhydrous gel. In some embodiments, the composition is in the form of a solution. In some embodiment, the composition is in the form of a hydrophobic gel.

In certain embodiments, the composition consists essentially of an α-hydroxy carboxylic acid ester and a second ester, wherein the α-hydroxy carboxylic acid ester and second ester are not the same. In some embodiments, the composition consists of an α-hydroxy carboxylic acid ester and a second ester, wherein the α-hydroxy carboxylic acid ester and second ester are not the same.

In certain embodiments, the ratio of α-hydroxy carboxylic acid ester to second ester is a range of 99:1 to 1:99 (e.g, 10:1, 5:1, 2:1, 1:1, 1:2, 1:5, or 1:10).

In another aspect, the invention is directed to a dosage form of a drug, the dosage form comprising a drug, and a composition comprising an α-hydroxy carboxylic acid ester and a second ester, wherein the two esters are not the same; and wherein less than 10% (e.g., by weight or volume) of the dosage form comprises fatty alcohols and/or alkyl lactyllactates and/or other related substances.

In certain embodiments, the second ester is a compound of formula (I):

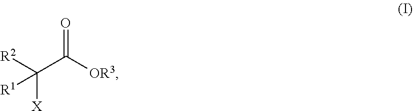

wherein
each R¹ is independently selected from the group consisting of: H, C₁-C₂₀ straight chained or branched alkyl, C₃-C₁₀ cycloalkyl, substituted C₁-C₂₀ alkyl, arylalkyl, aryl, substituted aryl and heteroaryl;
each R² is independently selected from the group consisting of: H and C₁-C₂₀ alkyl;
R³ is a C₁₂-C₄₀ alkyl; and

### X is NR¹R¹ or OR¹;

In some embodiments, the α-hydroxy carboxylic acid ester is a compound of formula (II):

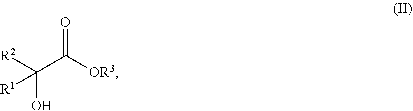

wherein R¹, R² and R³ are as defined in formula (I).

In some embodiments, the second ester is a compound of formula (II):

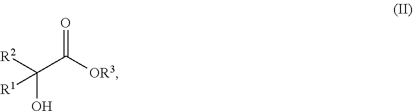

wherein R¹, R² and R³ are as defined in formula (I).

In some embodiments, the α-hydroxy carboxylic acid ester and the second esters are compounds of formula (II):

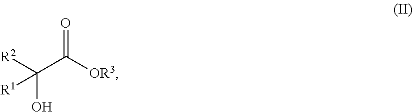

wherein R¹, R² and R³ are as described in formula (I).

In certain embodiments, the dosage form comprises less than 5% (e.g., by weight or volume) of the composition comprises fatty alcohols and/or alkyl lactyllactate and/or other related substances. In some embodiments, the dosage form comprises less than 3% (e.g., by weight or volume) of the composition comprises fatty alcohols and/or alkyl lactyllactate and/or other related substances. In some embodiments, the dosage form comprises less than 1% (e.g., by weight or volume) of the composition comprises fatty alcohols and/or alkyl lactyllactate and/or other related substances. In some embodiments, the dosage form comprises less than 0.1% (e.g., by weight or volume) of the composition comprises fatty alcohols and/or alkyl lactyllactate and/or other related substances. In some embodiments, the dosage form comprises less than 0.01% (e.g., by weight or volume) of the composition comprises fatty alcohols and/or alkyl lactyllactate and/or other related substances. In some embodiments, the dosage form comprises less than 0.001% (e.g., by weight or volume) of the composition comprises fatty alcohols and/or alkyl lactyllactates and/or other related substances.

In certain embodiments, the dosage form is substantially free of fatty alcohols and/or alkyl lactyllactates and/or other related substances.

In some embodiments, the dosage form is a topical dosage form. In some embodiments, the dosage form is in the form of an oil-in-water emulsion. In some embodiments, the dosage form is in the form of a water-in-oil emulsion. In some embodiments, the dosage form is in the form of a thickened aqueous gel. In some embodiments, the dosage form is in the form of a hydrophilic gel. In some embodiments, the dosage form is in the form of an anhydrous gel. In some embodiments, the dosage form is in the form of a solution. In some embodiments, the dosage form is in the form of a hydrophobic gel. In some embodiments, the dosage form is mounted on to a patch.

In some embodiments, the dosage form is configured to release the drug so as to maintain a desired blood level over a desired time period.

In another aspect, the invention is directed to a method of administering a drug to a subject, the method comprising administering to the surface of a subject a composition comprising a drug, an α-hydroxy carboxylic acid ester and a second ester, wherein the two esters are not the same, and wherein less than 10% (e.g., by weight or volume) of the composition comprises comprises fatty alcohols and/or alkyl lactyllactates and/or other related substances.

In certain embodiments, the second ester is a compound of formula (I):

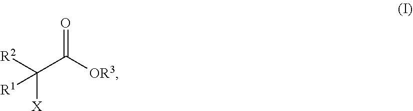

wherein
each R¹ is independently selected from the group consisting of: H, C₁-C₂₀ straight chained or branched alkyl, C₃-C₁₀ cycloalkyl, substituted C₁-C₂₀ alkyl, arylalkyl, aryl, substituted aryl and heteroaryl;
each R² is independently selected from the group consisting of: H and C₁-C₂₀ alkyl;
R³ is a C₁₂-C₄₀ alkyl; and

### X is NR¹R¹ or OR¹;

In some embodiments, the α-hydroxy carboxylic acid ester is a compound of formula (II):

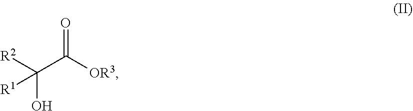

wherein R¹, R² and R³ are as defined in formula (I).

In some embodiments, the second ester is a compound of formula (II):

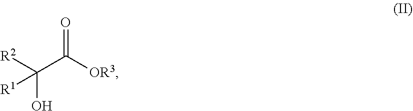

wherein R¹, R² and R³ are as defined in formula (I).

In some embodiments, the α-hydroxy carboxylic acid ester and the second esters are compounds of formula (II):

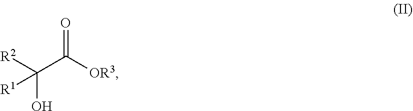

wherein R¹, R² and R³ are as described in formula (I).

In certain embodiments, the surface of a subject is a mucous membrane. In some embodiments, the surface of a subject is the skin.

In certain embodiments, the composition is administered topically. In some embodiments, the composition is administered bucally. In some embodiments, the composition is administered vaginally. In some embodiments, the composition is administered nasally. In some embodiments, the composition is administered via a patch.

In some embodiments, the composition is mounted onto a patch which adheres to the surface of a subject.

In certain embodiments, the method allows for systemic delivery of a drug. In some embodiments, the method allows for local delivery of a drug. In some embodiments, the composition allows for control of the permeation rate (e.g., independent of the drug). In some embodiments, the composition allows for control of the depth of penetration.

In another aspect, the invention is directed to a method of making a composition comprising a plurality of esters, the method comprising:

providing an α-hydroxy carboxylic acid ester with a purity of at least 90%;

providing a second ester with a purity of at least 90%; and

combining said first and second esters; wherein

the first and second esters are not the same.

In certain embodiments, the second ester is a compound of formula (I):

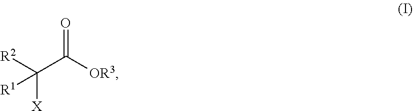

wherein
each R¹ is independently selected from the group consisting of: H, C₁-C₂₀ straight chained or branched alkyl, C₃-C₁₀ cycloalkyl, substituted C₁-C₂₀ alkyl, arylalkyl, aryl, substituted aryl and heteroaryl;
each R² is independently selected from the group consisting of: H and C₁-C₂₀ alkyl;
R³ is a C₁₂-C₄₀ alkyl; and

### X is NR¹R¹ or OR¹;

In some embodiments, the α-hydroxy carboxylic acid ester is a compound of formula (II):

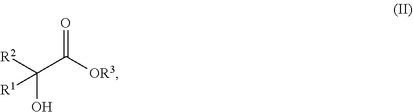

wherein R¹, R² and R³ are as defined in formula (I).

In some embodiments, the second ester is a compound of formula (II):

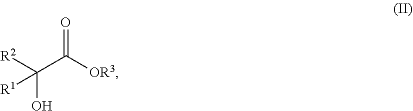

wherein R¹, R² and R³ are as defined in formula (I).

In some embodiments, the α-hydroxy carboxylic acid ester and the second esters are compounds of formula (II):

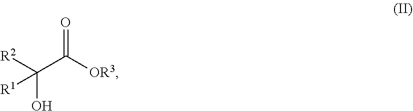

wherein R¹, R² and R³ are as described in formula (I).

In certain embodiments, the composition further comprises a third ester. In some embodiments, the composition further comprises a fourth ester. In some embodiments, the composition further comprises a fifth ester. In some embodiments, the composition comprises more than five or more esters.

In certain embodiments, the α-hydroxy carboxylic acid ester, the second ester and the third ester are not the same. In some embodiments, the α-hydroxy carboxylic acid ester, the second ester, the third ester and the fourth ester are not the same. In some embodiments, the α-hydroxy carboxylic acid ester, the second ester, the third ester, the fourth ester and the fifth are not the same.

In certain embodiments, the composition comprises providing a drug (e.g., a small molecule drug, a peptide or polypeptide, a protein or a hormone).

In certain embodiments, the α-hydroxy carboxylic acid ester has a purity of 90%. In some embodiments, the α-hydroxy carboxylic acid ester has a purity of 95%. In some embodiments, the α-hydroxy carboxylic acid ester has a purity of 97%. In some embodiments, the α-hydroxy carboxylic acid ester has a purity of 99%.

In certain embodiments, the second ester has a purity of 90%. In some embodiments, the second ester has a purity of 95%. In some embodiments, the second ester has a purity of 97%. In some embodiments, the second ester has a purity of 99%.

In certain embodiments, the third ester has a purity of 90%. In some embodiments, the third ester has a purity of 95%. In some embodiments, the third ester has a purity of 97%. In some embodiments, the third ester has a purity of 99%.

In certain embodiments, the fourth ester has a purity of 90%. In some embodiments, the fourth ester has a purity of 95%. In some embodiments, the fourth ester has a purity of 97%. In some embodiments, the fourth ester has a purity of 99%.

In certain embodiments, the fifth ester has a purity of 90%. In some embodiments, the fifth ester has a purity of 95%. In some embodiments, the fifth ester has a purity of 97%. In some embodiments, the fifth ester has a purity of 99%.

In another aspect, the invention is directed to a method of making a drug delivery device (e.g., a patch, cream, ointment or gel), comprising disposing composition comprising an α-hydroxy carboxylic acid ester and second ester on or in a said device; wherein

the first and second esters are not the same; and

wherein less than 10% (e.g., by weight or volume) of the device comprises fatty alcohols and/or alkyl lactyllactate and/or other related substances.

In certain embodiments, the second ester is a compound of formula (I):

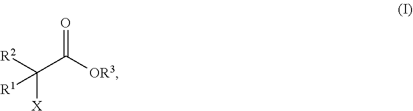

wherein
each R¹ is independently selected from the group consisting of: H, C₁-C₂₀ straight chained or branched alkyl, C₃-C₁₀ cycloalkyl, substituted C₁-C₂₀ alkyl, arylalkyl, aryl, substituted aryl and heteroaryl;
each R² is independently selected from the group consisting of: H and C₁-C₂₀ alkyl;
R³ is a C₁₂-C₄₀ alkyl; and

### X is NR¹R¹ or OR¹;

In some embodiments, the α-hydroxy carboxylic acid ester is a compound of formula (II):

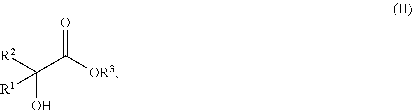

wherein R¹, R² and R³ are as defined in formula (I).

In some embodiments, the second ester is a compound of formula (II):

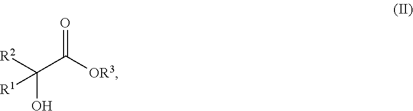

wherein R¹, R² and R³ are as defined in formula (I).

In some embodiments, the α-hydroxy carboxylic acid ester and the second esters are compounds of formula (II):

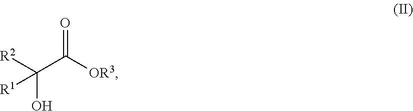

wherein R¹, R² and R³ are as described in formula (I).

In certain embodiments, less than 5% (e.g., by weight or volume) of the composition comprises fatty alcohols and/or alkyl lactyllactates and/or other related substances. In some embodiments, less than 3% (e.g., by weight or volume) of the composition comprises fatty alcohols and/or alkyl lactyllactates and/or other related substances. In some embodiments, less than 1% (e.g., by weight or volume) of the composition comprises fatty alcohols and/or alkyl lactyllactates and/or other related substances. In some embodiments, less than 0.1% (e.g., by weight or volume) of the composition comprises fatty alcohols and/or alkyl lactyllactates and/or other related substances.

In another aspect, the invention is directed to a method of providing a composition having a preselected value for a parameter rated to penetration, e.g., the rate migration through the skin, the method comprising:
selecting a value for the parameter;
selecting a mixture having of esters, the mixture having a relative amount of an α-hydroxy carboxylic acid ester and a second ester so as to provide for the selected value.

In certain embodiments, the second ester is a compound of formula (I):

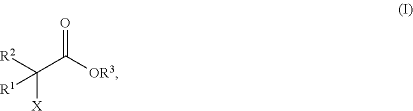

wherein
each R¹ is independently selected from the group consisting of: H, C₁-C₂₀ straight chained or branched alkyl, C₃-C₁₀ cycloalkyl, substituted C₁-C₂₀ alkyl, arylalkyl, aryl, substituted aryl and heteroaryl;
each R² is independently selected from the group consisting of: H and C₁-C₂₀ alkyl;
R³ is a C₁₂-C₄₀ alkyl; and

### X is NR¹R¹ or OR¹;

In some embodiments, the α-hydroxy carboxylic acid ester is a compound of formula (II):

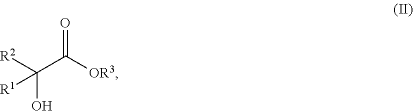

wherein R¹, R² and R³ are as defined in formula (I).

In some embodiments, the second ester is a compound of formula (II):

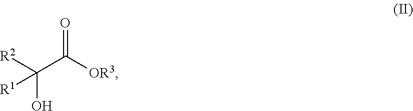

wherein R¹, R² and R³ are as defined in formula (I).

In some embodiments, the α-hydroxy carboxylic acid ester and the second esters are compounds of formula (II):

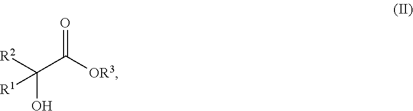

wherein R¹, R² and R³ are as described in formula (I).

In another aspect, the invention is directed to a device for sustained release of a drug comprising:

an optional adhesive element;

an optional drug storage element;

a drug;

an α-hydroxy carboxylic acid ester and a second ester, wherein the first and second esters are not the same, and wherein less than 10% (e.g., by weight or volume) of the device comprises fatty alcohols and/or alky lactyllactate and/or other related substances.

In certain embodiments, the second ester is a compound of formula (I):

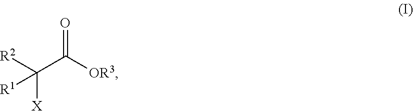

wherein
each R¹ is independently selected from the group consisting of: H, C₁-C₂₀ straight chained or branched alkyl, C₃-C₁₀ cycloalkyl, substituted C₁-C₂₀ alkyl, arylalkyl, aryl, substituted aryl and heteroaryl;
each R² is independently selected from the group consisting of: H and C₁-C₂₀ alkyl;
R³ is a C₁₂-C₄₀ alkyl; and

### X is NR¹R¹ or OR¹;

In some embodiments, the α-hydroxy carboxylic acid ester is a compound of formula (II):

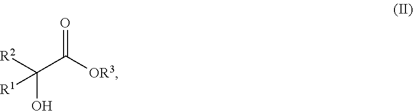

wherein R¹, R² and R³ are as defined in formula (I).

In some embodiments, the second ester is a compound of formula (II):

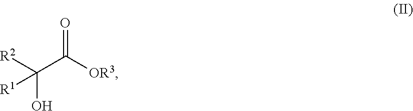

wherein R¹, R² and R³ are as defined in formula (I).

In some embodiments, the α-hydroxy carboxylic acid ester and the second esters are compounds of formula (II):

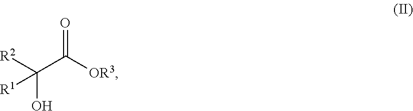

wherein R¹, R² and R³ are as described in formula (I).

In certain embodiments, the α-hydroxy carboxylic acid ester has a purity of at least 90%. In some embodiments, the α-hydroxy carboxylic acid ester has a purity of at least 95%. In some embodiments, the α-hydroxy carboxylic acid ester has a purity of at least 97%. In some embodiments, the α-hydroxy carboxylic acid ester has a purity of at least 99%.

In certain embodiments, the second ester has a purity of at least 90%. In some embodiments, the second ester has a purity of at least 95%. In some embodiments, the second ester has a purity of at least 97%. In some embodiments, the second ester has a purity of at least 99%.

### BRIEF DESCRIPTION OF THE FIGURES

FIG. 1a illustrates the % permeation of Lidocaine through EPIDERM 200™ cultured tissue for the lidocaine anhydrous formulations of example 1 over a 60 minute period.

FIG. 1b illustrates the % permeation of Lidocaine through EPIDERM 200™ cultured tissue for the lidocaine anhydrous formulations of example 1 over a 3 hour period.

FIG. 2a illustrates the % permeation of Lidocaine through EPIDERM 200™ cultured tissue for the lidocaine hydroalcoholic formulations of example 2 over a 60 minute period.

FIG. 2b illustrates the % permeation of Lidocaine through EPIDERM 200™ cultured tissue for the lidocaine hydroalcoholic formulations of example 2 over a 3 hour period.

FIG. 3a illustrates the % permeation of Ibuprofen through EPIDERM 200™ cultured tissue for the ibuprofen anhydrous formulations of example 3 over a 1 hour period.

FIG. 3b illustrates the % permeation of Ibuprofen through EPIDERM 200™ cultured tissue for the ibuprofen anhydrous formulations of example 3 over a 6 hour period.

FIG. 4a illustrates the % permeation of Ibuprofen through EPIDERM 200™ cultured tissue for the ibuprofen anhydrous formulations of example 4 over a 1 hour period.

FIG. 4b illustrates the % permeation of Ibuprofen through EPIDERM 200™ cultured tissue for the ibuprofen anhydrous formulations of example 4 over a 6 hour period.

### DETAILED DESCRIPTION

### Definitions

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 carbon atoms in it. The term “haloalkyl” refers to an alkyl in which one or more hydrogen atoms are replaced by halo, and includes alkyl moieties in which all hydrogens have been replaced by halo, e.g., perfluoroalkyl. The terms “arylalkyl” or “aralkyl” refer to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups.

The term “alkylene” refers to a divalent alkyl, e.g., —CH₂—, —CH₂CH₂—, and —CH₂CH₂CH₂—.

The term “alkenyl” refers to a straight or branched hydrocarbon chain containing 2-12 carbon atoms and having one or more double bonds. Examples of alkenyl groups include, but are not limited to, allyl, propenyl, 2-butenyl, 3-hexenyl and 3-octenyl groups. One of the double bond carbons may optionally be the point of attachment of the alkenyl substituent. The term “alkynyl” refers to a straight or branched hydrocarbon chain containing 2-12 carbon atoms and characterized in having one or more triple bonds. Examples of alkynyl groups include, but are not limited to, ethynyl, propargyl, and 3-hexynyl. One of the triple bond carbons may optionally be the point of attachment of the alkynyl substituent.

The term “aryl” refers to an aromatic monocyclic, bicyclic, or tricyclic hydrocarbon ring system, wherein any ring atom capable of substitution can be substituted, e.g., by one or more substituents. Examples of aryl moieties include, but are not limited to, phenyl, naphthyl, and anthracenyl.

The term “arylalkyl” or the term “aralkyl” refers to alkyl substituted with an aryl. Exemplary aralkyls include but are not limited to benzyl and phenethyl.

The term “cycloalkyl” as employed herein includes saturated cyclic, bicyclic, tricyclic, or polycyclic hydrocarbon groups having 3 to 12 carbons. Any ring atom can be substituted, e.g., by one or more substituents. The cycloalkyl groups can contain fused rings. Fused rings are rings that share a common carbon atom. Examples of cycloalkyl moieties include, but are not limited to, cyclopropyl, cyclohexyl, methylcyclohexyl, adamantyl, and norbornyl.

The term “emollient” is a hydrophobic material that provides softness, lubricity and smoothness to the skin and often forms a thin occlusive film which increases hydration by reducing transepidermal water loss (TEWL).

The “enantiomeric excess” or “% enantiomeric excess” of a composition can be calculated using the equation shown below. In the example shown below a composition contains 90% of one enantiomer, e.g., the S enantiomer, and 10% of the other enantiomer, i.e., the R enantiomer.

ee=(90−10)/100=80%.

Thus, a composition containing 90% of one enantiomer and 10% of the other enantiomer is said to have an enantiomeric excess of 80%. Some of the compositions described herein contain an enantiomeric excess of at least 50%, 75%, 90%, 95%, or 99% of Compound 1 (the S-enantiomer). In other words the compositions contain an enantiomeric excess of the S enantiomer over the R enantiomer.

The term “heterocyclyl” refers to a nonaromatic 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, Si, P or S, e.g., carbon atoms and 1-3, 1-6, or 1-9 heteroatoms of N, O, Si, P or S if monocyclic, bicyclic, or tricyclic, respectively. The heteroatom may optionally be the point of attachment of the heterocyclyl substituent. Any ring atom can be substituted, e.g., by one or more substituents. The heterocyclyl groups can contain fused rings. Fused rings are rings that share a common carbon atom. Examples of heterocyclyl include, but are not limited to, tetrahydrofuranyl, tetrahydropyranyl, piperidinyl, morpholino, pyrrolinyl, pyrimidinyl, quinolinyl, and pyrrolidinyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, Si, P or S, e.g., carbon atoms and 1-3, 1-6, or 1-9 heteroatoms of N, O, Si, P or S if monocyclic, bicyclic, or tricyclic, respectively. Any ring atom can be substituted, e.g., by one or more substituents.

The term “humectant” is a polar hygroscopic material that increases hydration by drawing water from the environment to help retain water in the skin's upper layers.

The term “lesion” refers to an abnormal condition of a tissue (e.g., skin and/or mucous membrane) caused by a microbial (e.g., bacterial, viral and/or fungal) infection.

The term “moisturizer” refers to a material that will increase the level of hydration of skin, mucous membrane, wound, lesion or scab.

The term “purity” refers to the degree to which a substance is undiluted or unmixed with extraneous material and is typically expressed as a percentage.

The term “substantially free” when referring to a compound or composition described herein means that there is less than 20% (by weight) of the designated compound or by-product (e.g., a saturated alcohol starting material) present, more preferably, there is less than 10% (by weight) of the designated compound or by-product, more preferably, there is less than 9% (by weight) of the designated compound or by-product, more preferably, there is less than 8% (by weight) of the designated compound or by-product, more preferably, there is less than 7% (by weight) of the designated compound or by-product, more preferably, there is less than 6% (by weight) of the designated compound or by-product, more preferably, there is less than 5% (by weight) of the designated compound or by-product, more preferably, there is less than 4% (by weight) of the designated compound or by-product, more preferably, there is less than 3% (by weight) of the designated compound or by-product, more preferably, there is less than 2% (by weight) of the designated compound or by-product, and most preferably, there is less than 1% (by weight) of the designated compound or by-product.

The term “substituents” refers to a group “attached” to a alkyl, cycloalkyl, alkenyl, alkynyl, heterocyclyl, heterocycloalkenyl, cycloalkenyl, aryl, or heteroaryl group at any atom of that group. Suitable substituents include, without limitation, alkyl, e.g., C1, C2, C3, C4, C5, C6, C7, C8, C9, C10, C11, C12 straight or branched chain alkyl, cycloalkyl, haloalkyl, e.g., perfluoroalkyl such as CF₃, aryl, heteroaryl, aralkyl, heteroaralkyl, heterocyclyl, alkenyl, alkynyl, cycloalkenyl, heterocycloalkenyl, alkoxy, haloalkoxy, e.g., perfluoroalkoxy such as OCF₃, halo, hydroxy, carboxy, carboxylate, cyano, nitro, amino, alkyl amino, SO₃H, sulfate, phosphate, methylenedioxy e.g., —O—CH₂—O—, ethylenedioxy, oxo, thioxo, e.g., C═S, imino, e.g., alkyl, aryl, aralkyl, S(O)ₙalkyl, S(O)ₙaryl, S(O)ₙheteroaryl, S(O)ₙheterocyclyl, i.e., wherein is an integer between 0 and 2, amine, e.g., mono-, di-, alkyl, cycloalkyl, aralkyl, heteroaralkyl, aryl, heteroaryl, and combinations thereof, ester, e.g., alkyl, aralkyl, heteroaralkyl, aryl, heteroaryl, amide, e.g., mono-, di-, alkyl, aralkyl, heteroaralkyl, aryl, heteroaryl, and combinations thereof, sulfonamide, e.g., mono-, di-, alkyl, aralkyl, heteroaralkyl, and combinations thereof. In one aspect, the substituents on a group are independently any one single, or any subset of the aforementioned substituents. In another aspect, a substituent may itself be substituted with any one of the above substituents.

The term “wound” refers to an injury to a subject which involves a break in the normal skin or mucosal tissue barrier exposing tissue below, which is caused by, for example, lacerations, surgery, burns, damage to underlying tissue such as pressure sores, poor circulation and the like. Wounds are understood to include both acute and chronic wounds.

### Compounds

In general, the compounds described herein may be an α-hydroxycarboxylic acid ester or may be any other ester. In one aspect, the compounds utilized in the composition of the present application are represented by formula (I):

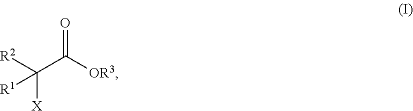

wherein R¹, R², R³ and X are as represented herein. In another aspect, the compounds utilized in the compositions of the present application are represented by formula (II):

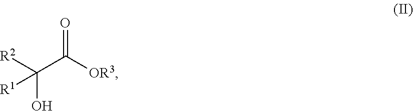

wherein R¹, R² and R³ are as defined in formula (I).

In one aspect, the invention features a composition containing a racemic mixture of a compound. In one aspect, the invention features a composition containing an enantiomeric excess (ee) of a compound (e.g., a compound of formula (I) or (II)). For example, the composition can contain an ee of at least 10%, 50%, 75%, 90%, 95%, or 99%. In another aspect, the invention features a composition containing a racemic mixture (e.g., less than 10% ee) of the compound described herein (e.g., the compounds of formulas (I) and (II)). The compounds employed in the compositions described herein (e.g., a compound of formula (I) or (II)) can be made using a variety of synthetic techniques. In one aspect, a compound described herein (e.g., a compound of formula (I) or (II)) can be made as illustrated in pending U.S. application Ser. No. 11/251,738, which is hereby incorporated by reference in its entirety.

A drug or therapeutic agent employed or described herein can also be employed in the form of a prodrug. Prodrugs of the drug and/or therapeutic agent described herein are compounds that readily undergo chemical changes under physiological conditions to provide the required drug/therapeutic agent. Additionally, prodrugs can be converted to the drugs/therapeutic agents employed in the present invention by chemical or biochemical methods in an ex vivo environment. For example, prodrugs can be slowly converted to the drug/therapeutic agents employed in the present invention when placed in a transdermal patch reservoir with a suitable enzyme or chemical reagent.

A compound of the present invention can exist in an unsolvated form as well as a solvated form, including hydrated forms. In general, the solvated forms are equivalent to unsolvated forms and are encompassed within the scope of the present invention. Certain compounds of the present invention may exist in multiple crystalline or amorphous forms termed polymorphic forms. In general, all physical forms are of use in the methods contemplated by the present invention and are intended to be within the scope of the present invention. “Compound or a pharmaceutically acceptable salt, hydrate, polymorph or solvate of a compound” intends the inclusive meaning of “or”, in that materials meeting more than one of the stated criteria included, for example, a material that is both a salt and a solvate is encompassed.

A compound described herein can be in the form of a metabolite. A metabolite may be a compound that is related to a compound described herein, as a form of such compound obtained in a human or animal body by action of the body on the administered form of the compound. For example, a metabolite may be a de-methylated analogue of a compound bearing a methyl group, which is obtained in the body after administration of the methylated compound as a result of action by the body on the methylated compound. A metabolite may also be a carboxylic-acid containing compound, which is obtained in the body after administration of the corresponding ester as a result of action by the body on the ester-containing compound.

### Compositions of the Invention

The inventors have discovered novel ways of controlling the delivery of a therapeutic agents through the skin or mucousal membrane of a subject. For example, the inventors have discovered that the use of a plurality of esters, e.g., alpha hydroxy carboxylic acid esters or a combination of an alpha hydroxy carboxylic acid ester with an additional ester such as an othisalic ester, are effective in delivery of a therapeutic agent to a subject through the skin or mucous membrante of the subject. Moreover, the inventors have discovered that varying the relative amount of the esters can result in a change in the relative rate of delivery of a therapeutic agent through the skin of a subject. Applicants' ability to provide for each ester in ultra pure form provides a unique ability to “dial-in” a delivery rate with specific control, due to Applicants' ability to maintain tight control over the components in each formulation. These compositions also allow for delivery of a drug and/or therapeutic agent independent of the rate at which the drug and/or therapeutic agent enters the body.

The present invention features pharmaceutical compositions including any of the ester compounds described herein (e.g., a compound of formula (I) or (II)), either alone or in combination with one or more excipients. In some embodiments, the pharmaceutical composition is a composition that can be administered topically. In some embodiments, the composition is a composition that can be administered bucally, vaginally, mucosally, or nasally (e.g., intranasally), intranasally or via patch. In some embodiments, the composition is a solid composition, for example, a lyophilisate, which can be further processed prior to administering the composition to a subject, for example, the solid composition can be further processed to form a liquid composition such as a solution.

The compositions described herein, e.g., a composition including esters described herein, can be used as a topical drug delivery composition. These compositions may also include one or more drugs and/or active agents used to treat a variety of indications including, but not limited to viral infections, pain, asthma, skin diseases and microbial infections. In some embodiments, the compositions described herein consist essentially of esters described herein (e.g., a compound of formulas (I) or (II)). Certain compositions may also include one or more external analgesics and/or one or more moisturizers.

Certain compositions described herein adhere well to bodily tissues (e.g., mammalian tissues such as skin and mucosal tissues) and thus are very effective topically. Certain methods involve topical application, particularly to skin and mucous membranes. The compositions described herein, e.g., a composition including an esters described herein, may be used for treating or preventing pain, asthma, an infection caused by a virus, a microbial infection and various skin diseases.

Exemplary compositions may include one or more additional excipients. Said excipients may be selected from, but not limited to moisturizers, skin protectants, enhancer components, surfactants, and thickeners.

### Moisturizers

Compositions of the present invention may include a moisturizer to increase the level of hydration of the skin, mucous membrane, wound, lesion or scab. The moisturizer can be a hydrophilic material including humectants or it can be a hydrophobic material including emollients. A humectant is a polar hygroscopic material that increases hydration by drawing water from the environment to help retain water in the skin's upper layers. An emollient is a hydrophobic material that provides softness, lubricity and smoothness to the skin and often forms a thin occlusive film that increases hydration by reducing transepidermal water loss (TEWL). Exemplary hydrophilic moisturizers include, but are not limited to, water, polyhydric alcohols, lower alkyl ethers, N-methylpyrrolidone, lower alkyl esters, urea, amino acids, ethoxylated amides, sodium pyrrolidone carboxylic acid, and the lower monohydroxy alcohols and hydroxy acids discussed below as enhancers, as well as combinations thereof. Thus, a lower monohydroxy alcohol can function as both a hydrophilic compound and an enhancer. Preferably, the hydrophilic components include polyhydric alcohols, lower alkyl ethers, and short chain esters. More preferably, the hydrophilic components include polyhydric alcohols.

Exemplary hydrophobic moisturizers include, but are not limited to, short chain (i.e., C1-C6) alkyl or (C6-C12) aryl esters of long (i.e., C8-C36) straight or branched chain alkyl or alkenyl alcohols or acids and polyethoxylated derivatives of the alcohols; short chain (i.e., C1-C6) alkyl or (C6-C12) aryl esters of (C4-C12) diacids or (C4-C12) diols optionally substituted in available positions by —OH; (C2-C18) alkyl or (C6-C12) aryl esters of glycerol, pentaerythritol, ethylene glycol, propylene glycol, as well as polyethoxylated derivatives of these; (C12-C22) alkyl esters or (C12-C22) ethers of polypropylene glycol; (C12-C22) alkyl esters or (C12-C22) ethers of polypropylene glycol/polyethylene glycol copolymer; and polyether polysiloxane copolymers. Additional examples of hydrophobic components include cyclic dimethicones, including volatile cyclic silicones such as D4 and D5, polydialkylsiloxanes, polyaryl/alkylsiloxanes, silicone copolyols, cocoa butter, beeswax, jojoba oil, lanolin and derivatives, long chain (i.e., C8-C36) alkyl and alkenyl esters of long (i.e., C8-C18) straight or branched chain alkyl or alkenyl alcohols or acids, long chain (i.e., C8-C36) alkyl and alkenyl amides of long straight or branched chain (i.e., C8-C36) alkyl or alkenyl amines or acids; hydrocarbons including straight and branched chain alkanes and alkenes such as isoparafins (e.g., isooctane, isododecane, isooctadecane, etc.), squalene, and mineral oil, polysiloxane polyalkylene copolymers, dialkoxy dimethyl polysiloxanes; (C12-C22) alkyl and (C12-C22) alkenyl alcohols, and petroleum derived alkanes such as isoparafins, petrolatum, petrolatum USP, as well as refined natural oils (especially NF or USP grades) such as olive oil NF, cotton seed oil, castor oil, peanut oil, corn oil, seasame oil, safflower oil, soybean oil, sunflower oil and the like, and blends thereof. In certain preferred embodiments, the hydrophobic components useful in the compositions of the present invention include those selected from the group consisting of petrolatum USP and short chain (i.e., C1-C6) alkyl or (C6-C12) aryl esters of long (i.e., C8-C36) straight or branched chain alkyl or alkenyl alcohols or acids and polyethoxylated derivatives of the alcohols; short chain (i.e., C1-C6) alkyl or (C6-C12) aryl esters of (C4-C12) diacids or (C4-C12) diols optionally substituted in available positions by —OH (such as diisopropyladipate, diisopropylsebacate); (C1-C9) alkyl or (C6-C12) aryl esters of glycerol, pentaerythritol, ethylene glycol, propylene glycol (such as glyceryl tricaprylate/caprate); and mixtures thereof.

### Skin Protectants

Compositions of the present invention may also include a skin protectant. Certain materials including some humectants or emollients are also useful at providing safe and effective skin protection. When used in the appropriate amount they temporarily protect injured or exposed skin or mucous membrane surfaces from harmful stimuli and may help provide relief to such surfaces. Information concerning safe and effective skin protectants is provided in the Proposed Final Rulemaking for Fever Blister and Cold Sore Treatment Drug Products in the Skin Protectant Drug Products for Over-the-counter Human Use Monograph, published by the United States Food and Drug Administration in the Federal Register, Volume 51, Number 21, Jan. 31, 1990, pages 3362 to 3370.

### Enhancer Component

Compositions of the present invention may optionally include an enhancer to enhance the antimicrobial activity (e.g., against gram negative bacteria). The enhancer component may include but is not limited to an alpha-hydroxy acid, a beta-hydroxy acid, other carboxylic acids, a (C1-C4) alkyl carboxylic acid, a (C6-C12) aryl carboxylic acid, a (C6-C12) aralkyl carboxylic acid, a (C6-C12) alkaryl carboxylic acid, a phenolic compound (such as certain antioxidants and parabens), a (C1-C10) monohydroxy alcohol, a chelating agent, or a glycol ether (i.e., ether glycol) and/or mixtures thereof.

### Surfactants

Compositions of the present invention optionally may include one or more surfactants to emulsify the composition and to help wet the surface and/or to aid in contacting the microorganisms. In general, a “surfactant” refers to an amphiphile (i.e., a molecule possessing both polar and nonpolar regions which are covalently bound) capable of reducing the surface tension of water and/or interfacial tension between water and an immiscible liquid. Surfactants that may be employed in the present compositions include, but are not limited to include soaps, detergents, emulsifiers, surface active agents, and the like. The surfactant can be cationic, anionic, nonionic, or amphoteric. In preferred embodiments, the surfactant includes poloxamer, ethoxylated stearates, sorbitan oleates, high molecular weight crosslinked copolymers of acrylic acid and a hydrophobic comonomer, and cetyl and stearyl alcohols as cosurfactants.

### Thickeners

Compositions of the present invention may also include thickeners that are soluble, swellable, or insoluble organic polymeric thickeners such as natural and synthetic polymers including polyacrylic acids, poly(N-vinyl pyrrolidones), cellulosic derivatives, silicon elastomers and xanthan or guar gums or inorganic thickeners such as silica, fumed silica, precipitated silica, silica aerogel and carbon black, and the like; other particle fillers such as calcium carbonate, magnesium carbonate, kaolin, talc, titanium dioxide, aluminum silicate, diatomaceous earth, ferric oxide and zinc oxide, clays, and the like; ceramic microspheres or glass microbubbles; ceramic microspheres such as those available under the tradenames “ZEOSPHERES” or “Z-LIGHT” from 3M Company, St. Paul, Minn. and/or combinations thereof.

### Forms

The pharmaceutical compositions of this invention may be administered topically. Compositions suitable for topical administration may be in a variety of forms amenable to topical administration, each containing a predetermined amount of a compound of the invention(s) as an active ingredient. Generally, the compositions of this invention may be in one of the following forms:

A hydrophobic or hydrophilic ointment wherein the composition is formulated with a hydrophobic base (e.g., petroleum, thickened or gelled water-insoluble oils, etc.) and optionally having a minor amount of a water soluble phase. Hydrophilic ointments generally contain one or more surfactants or wetting agents.

An oil-in-water emulsion wherein the compositions described may be formulated in which the antiviral component is emulsified into an emulsion comprising a discrete phase of a hydrophobic component and a continuous aqueous phase that includes water and optionally one or more polar hydrophilic material(s) as well as salts, surfactants, emulsifiers and other components. These emulsions may include water soluble or water-swellable polymers as well as one or more emulsifiers that help to stabilize the emulsion. These emulsions generally have higher conductivity values, as disclosed in U.S. Pat. No. 7,030,203.

A water-in-oil emulsion wherein the compositions described herein may be formulated so that the antiviral components are incorporated into an emulsion that includes a continuous phase of a hydrophobic component and an aqueous phase that includes water and optionally one or more polar hydrophilic material(s) as well as salts or other components. These emulsions may include oil-soluble or oil-swellable polymers as well as one or more emulsifier(s) that help to stabilize the emulsion.

Thickened aqueous gels refer to systems including an aqueous phase which has been thickened by suitable natural, modified natural or synthetic polymers as described herein. Alternatively, the thickened aqueous gels can be thickened using suitable polyethoxylated alkyl chain surfactants that effectively thicken the composition as well as other non-ionic, cationic or anionic emulsifier systems.

Hydrophobic gels refers to systems in which the continuous phase includes at least one hydrophobic components (e.g., a hydrophobic polymer) and is substantially free of water. Hydrophilic gels refer to systems in which the continuous phase includes at least one water soluble or water dispersible hydrophilic component other than water. The formulations may optionally also contain water up to 20% by weight. Higher levels may be suitable in some compositions. Suitable hydrophilic components include one or more glycols such as polyols such as glycerin, propylene glycol, butylene glycols, polyethylene glycols (PEGS), random or block copolymers of ethylene oxide, propylene oxide, and/or butylene oxide, polyalkoxylated surfactants having one or more hydrophobic moieties per molecule, silicone copolyols, as well as combinations thereof. One of ordinary skill in the art will recognize and understand that the level of ethoxylation should be sufficient to render the hydrophilic component water soluble or water dispersible at 23° C. In most embodiments, the water content is less than 20%, preferably less than 10% and preferably less than 5% by weight of the composition.

### EXAMPLES

General Procedures.

Commercial reagents were purchased from various sources as indicated below unless otherwise noted:

Carbopol® Ultrez 20 (acrylates/C10-30 alkyl acrylates crosspolymer): purchased from Labrizol (Product No. 0100763521).
Caprylic/capric triglyceride (Labrafac Lipophile WL 1349): purchased from Gattefosse (Product No. 117013/102809-02AE).
Cetyl lactate, 98.3%: supplied by Chemic Laboratories (Product No. CLI 3407-01).
Diethylene glycol monomethyl ether (DGME) (Carbitol®): purchased from Spectrum (WG0673).
Ethanol, 200 proof: Purchased from Spectrum (Product No. UK0558).
Ibuprofen: purchased from Sigma (Product No. 026H1368/REF060810-031).
Lauryl lactate, 98.9% (CHRYSTAPHYL®): supplied by Chemic Laboratories (Product No. CLI 4414-25-R&D blend).
Myristyl lactate, 96.4%: supplied by Chemic Laboratories (Product No. CLI 3369-18D).
Isododecane dimethicone/bis-isobutyl PPG-20 crosspolymer (EL-8050 ID Silicone Organic Elastomer Blend): purchased from Dow-Corning (Product No. 0005653647/RAW 102709-02U).
Octyl salicylate (Octisalate, USP): purchased from Spectrum (Product No. YV0632).
Phenyl trimethicone (556 Cosmetic Grade Fluid): purchased from Dow-Corning Squalene: purchased from Spectrum (Product No. YT0465).
Sunflower seed oil: purchased from Spectrum (Product No. YN1010/RAW122909-01T).
Silica silylate (VM-2270 Aerogel Fine Particles): purchased from Dow-Corning.
Triisopropanolamine, 95%: purchased from Sigma-Aldrich (Product No. 01721JI).

### Example 1

### Lidocaine (4%) Anhydrous Formulations

### General Preparation Procedure:

An appropriate amount of lidocaine was weighed in a mixing vessel. To the lidocaine was added caprylic triglyceride (Labrafac Lipophile WL 1349), octyl salicylate (Octisalate), phenyl trimethicone (556 Cosmetic Grade Fluid), Squalene and Sunflower seed oil in succession with stirring and/or sonication after each addition to provide a homogenous solution. In a separate mixing vessel, the appropriate amount of lauryl lactate (CHRYSTAPHYL®) (and/or other esters, e.g., myristyl lactate, cetyl lactate, stearyl lactate and/or lauryl mandelate) was weighed. To the second mixing vessel was added the lidocaine solution with sonication and/or stirring to provide a homogenouse solution. To the mixture was added isododecane dimethicone/bis-isobutyl PPG-20 crosspolymer (EL-8050 ID Silicone Organic Elastomer Blend). The resulting mixture was blended for 5-15 min. or until homogenous. To the mixture was subsequently added silica silylate (VM-2270 Aerogel Fine Particles). The resulting mixture was blended for 10-30 min. or until homogenous.

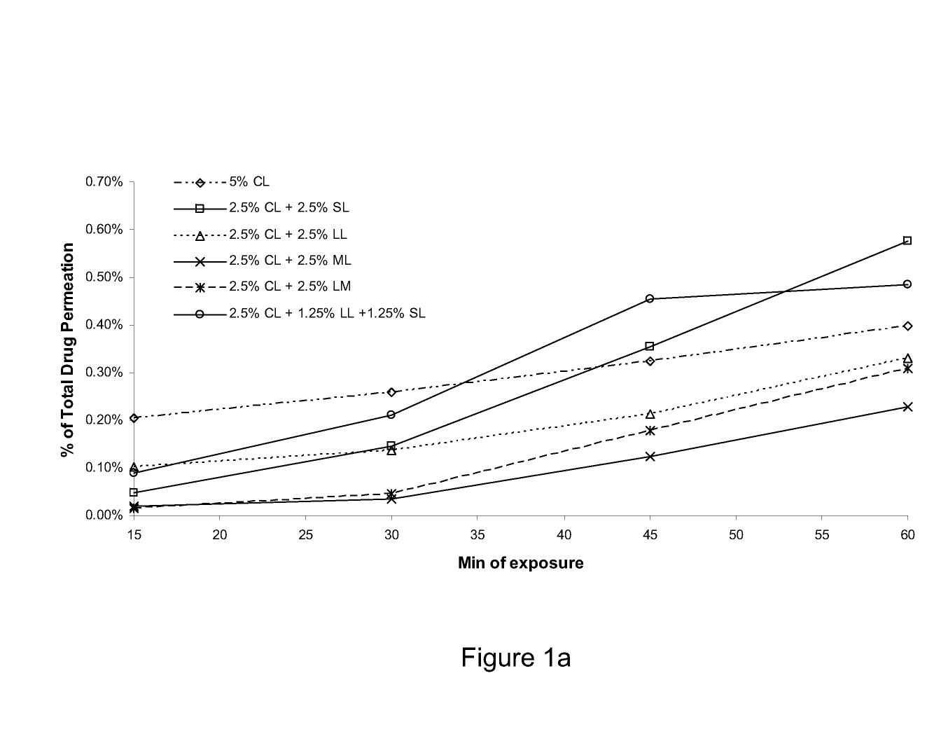

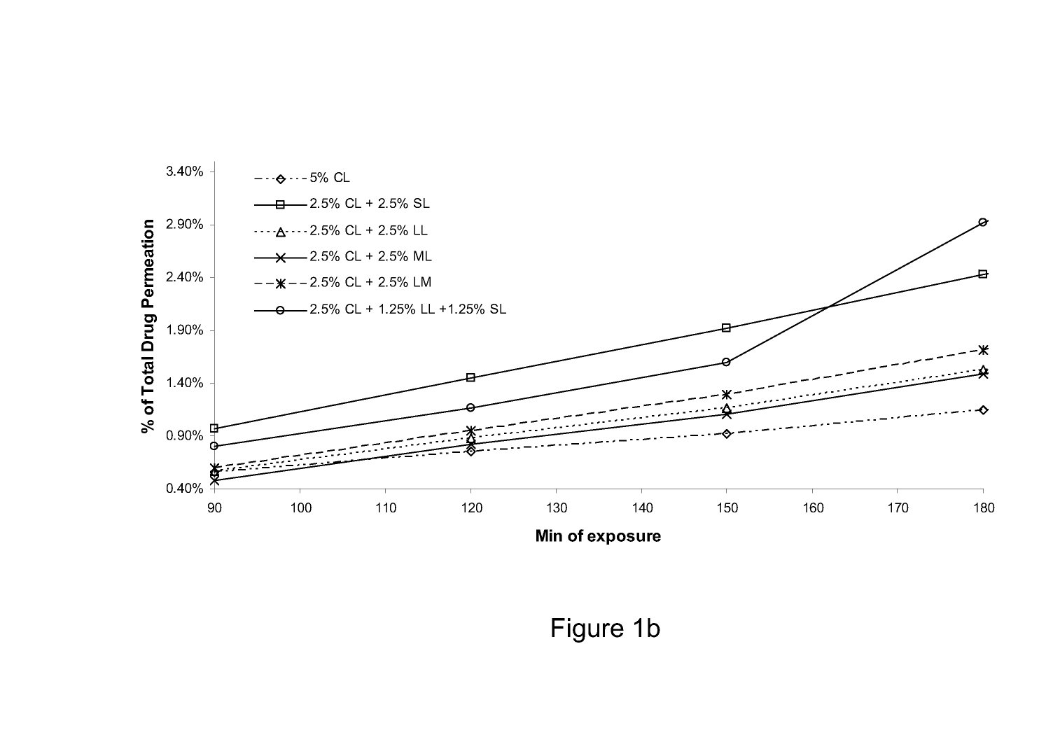

The above procedure can be used to produce a variety of formulations including a variety of ester mixtures. Some formulations produced by the above example are illustrated in Table 1 below. The % permeation of lidocaine through an Epiderm 200 Cultured Tissue for each of these formulations is illustrated in FIGS. 1a & b over a 60 minute period and a 3 hour period respectively.

TABLE 1

| | | | | | | Formulation |
|---|---|---|---|---|---|---|
| | | Formulation | Formulation | Formulation | Formulation | 2.5% CL + |
| | Formulation | 2.5% CL + | 2.5% CL + | 2.5% CL + | 2.5% CL + | 1.25% LL + |
| | 5% CL | 2.5% SL | 2.5% LL | 2.5% ML | 2.5% LM | 1.25% SL |
| | % w/w of Ingredients | | | | | |
| Lidocaine | 4.0% | 4.0% | 4.0% | 4.0% | 4.0% | 4.0% |
| Caprilic Capric | 8.0% | 8.0% | 8.0% | 8.0% | 8.0% | 8.0% |
| Triglyceride | | | | | | |
| Octisalate | 5.0% | 5.0% | 5.0% | 5.0% | 5.0% | 5.0% |
| Phenyl Trimethicone | 7.5% | 7.5% | 7.5% | 7.5% | 7.5% | 7.0% |
| Squalene | 2.0% | 2.0% | 2.0% | 2.0% | 2.0% | 2.0% |
| Sunflower Seed Oil | 2.0% | 2.0% | 2.0% | 2.0% | 2.0% | 2.0% |
| Cetyl lactate | 5.0% | 2.5% | 2.5% | 2.5% | 2.5% | 2.5% |
| Stearyl lactate | — | 2.5% | — | — | — | 1.27% |
| Lauryl lactate | — | — | 2.5% | — | — | 1.23% |
| Myristyl lactate | — | — | — | 2.5% | — | — |
| Lauryl Mandelate | — | — | — | — | 2.5% | — |
| Dow Corning EL-8050 | 66.4% | 66.4% | 66.4% | 66.4% | 66.4% | 66.4% |
| Dow Corning Aerogel | 0.1% | 0.1% | 0.1% | 0.1% | 0.1% | 0.1% |
| Total Formulation % | 100% | 100% | 100% | 100% | 100% | 100% |

### Example 2

### Lidocaine (4%) Hydroalcoholic Formulations

### General Preparation Procedure:

An appropriate amount of Lidocaine was weighed in a mixing vessel. To the Lidocaine was added ethanol with stirring and/or sonication to provide a homogenous solution. To the solution was added the appropriate amount of lauryl lactate (CHRYSTAPHYL®) (and/or other esters, e.g., myristyl lactate, cetyl lactate, stearyl lactate and/or lauryl mandelate) was weighed. To the resulting mixture was added water. The mixture was blended for 1-2 min. or until homogenous. To the resulting mixture was added the appropriate amount of carbomer with stirring. The mixture was stirred for an additional 15-60 min. or until homogenous. To the resulting mixture was added triethanolamine (Trolamine). The mixture was stirred until the homogenous.

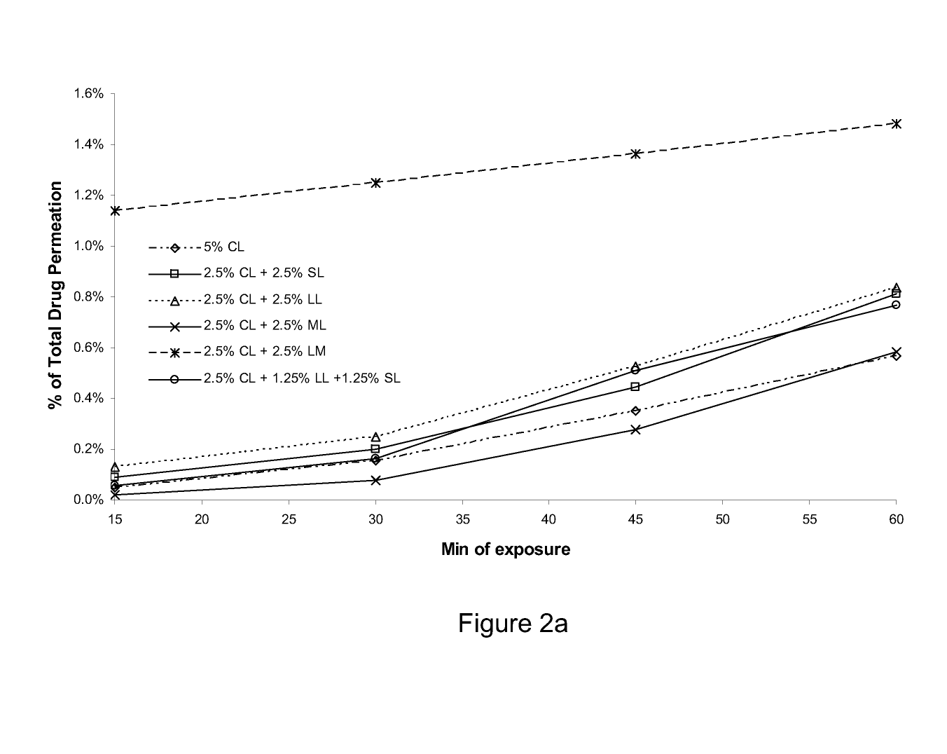

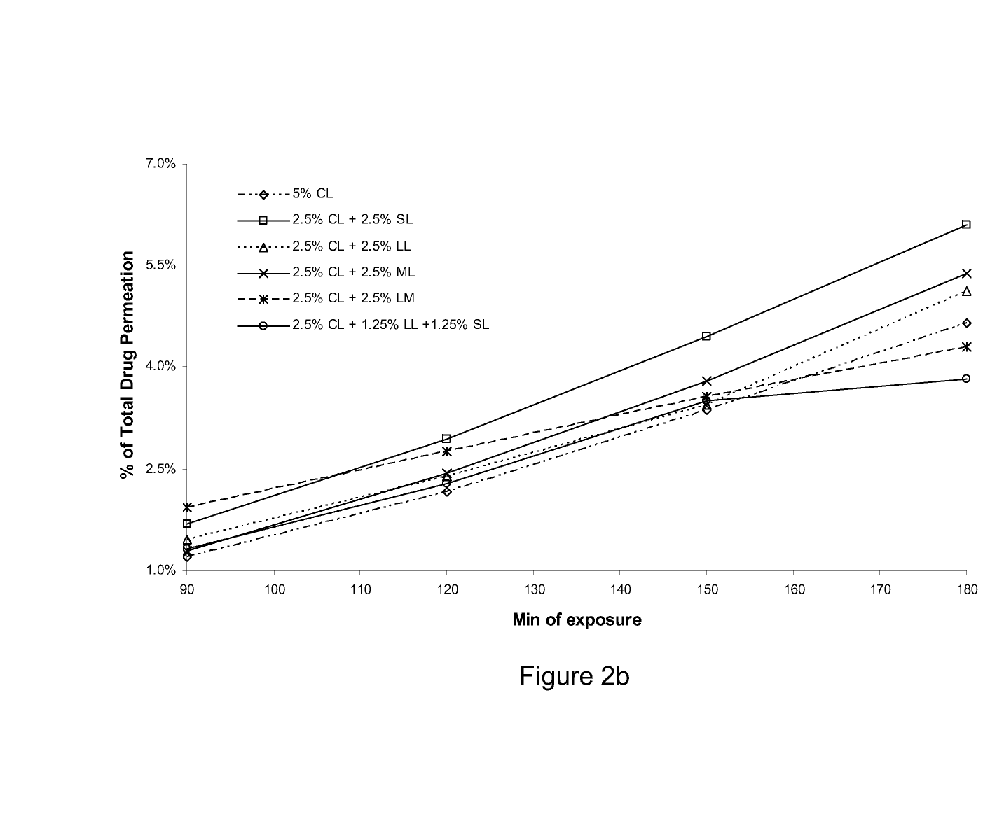

The above procedure can be used to produce a variety of hydroalcoholic formulations including a variety of ester mixtures. Some formulations produced by the above example are illustrated in Table 2 below. The % permeation of lidocaine through an Epiderm 200 Cultured Tissue for each of these formulations is illustrated in FIGS. 2a & b over a 60 minute period and a 3 hour period respectively.

TABLE 2

| | | | | | | Formulation |
|---|---|---|---|---|---|---|
| | | Formulation | Formulation | Formulation | Formulation | 2.5% CL + |
| | Formulation | 2.5% CL + | 2.5% CL + | 2.5% CL + | 2.5% CL + | 1.25% LL + |
| | 5% CL | 2.5% SL | 2.5% LL | 2.5% ML | 2.5% LM | 1.25% SL |
| | % w/w of Ingredients | | | | | |
| Lidocaine | 4.0% | 4.0% | 4.0% | 4.0% | 4.0% | 4.0% |
| EtOH | 70.0% | 70.0% | 70.0% | 70.0% | 70.0% | 70.0% |
| Cetyl lactate | 5.0% | 2.5% | 2.5% | 2.5% | 2.5% | 2.5% |
| Stearyl lactate | — | 2.5% | — | — | — | — |
| Lauryl lactate | — | — | 2.5% | — | — | 1.25% |
| Myristyl lactate | — | — | — | 2.5% | — | 1.25% |
| Lauryl Mandelate | — | — | — | — | 2.5% | — |
| Water | 19.9% | 19.9% | 19.9% | 19.9% | 19.9% | 19.9% |
| Carbomer 980 | 1.0% | 1.0% | 1.0% | 1.0% | 1.0% | 1.0% |
| Trolamine | 0.1% | 0.1% | 0.1% | 0.1% | 0.1% | 0.1% |
| Total Formulation % | 100% | 100% | 100% | 100% | 100% | 100% |

### Example 3

### Ibuprofen (5%) Anhydrous Formulations

### General Preparation Procedure

An appropriate amount of ibuprofen was weighed in a 20 mL scintillation vial. To the ibuprofen was added caprylic triglyceride (Labrafac Lipophile WL 1349), octyl salicylate (Octisalate), the appropriate amount of lauryl lactate (CHRYSTAPHYL®) (and/or other esters, e.g., myristyl lactate, cetyl lactate, stearyl lactate and/or lauryl mandelate) and diethylene glycol monomethyl ether (DGME) in succession. The reaction vial was then capped and the resulting mixture sonicated for 30 min. to provide a clear, homogenous solution. Squalene, Sunflower seed oil and phenyl trimethicone (556 Cosmetic Grade Fluid) were subsequently added in succession with mixing after each addition to provide a homogenous solution. To the mixture was added isododecane dimethicone/bis-isobutyl PPG-20 crosspolymer (EL-8050 ID Silicone Organic Elastomer Blend) with stirring until a clear, homogenous gel resulted.

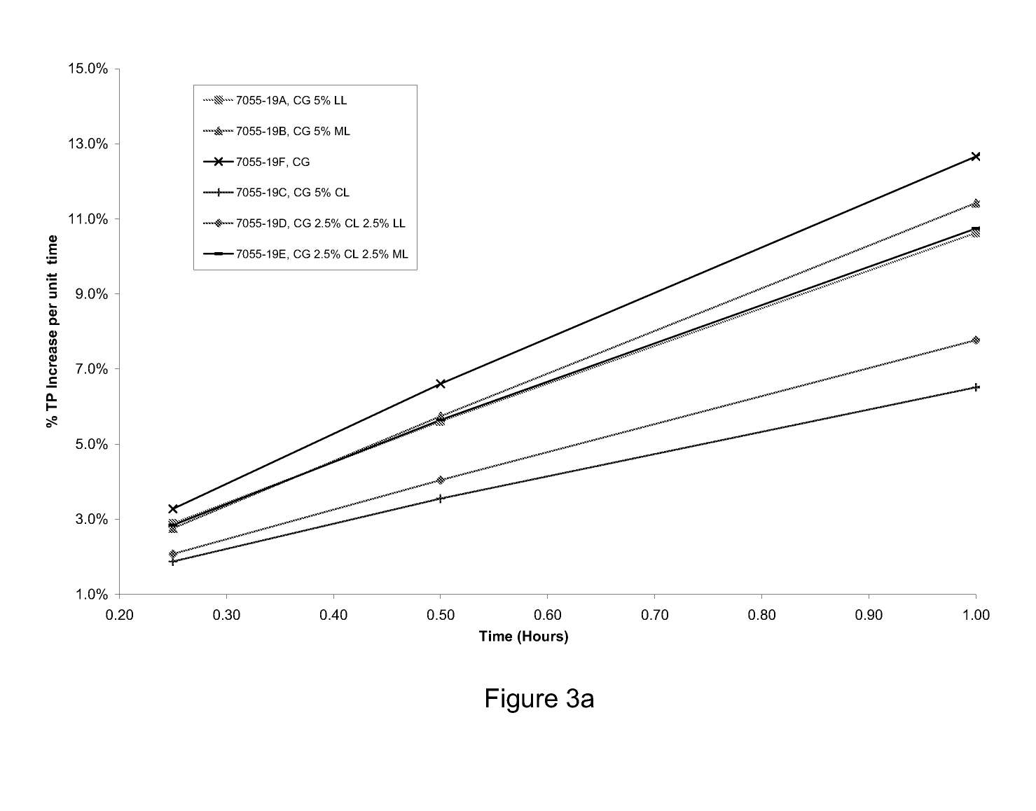

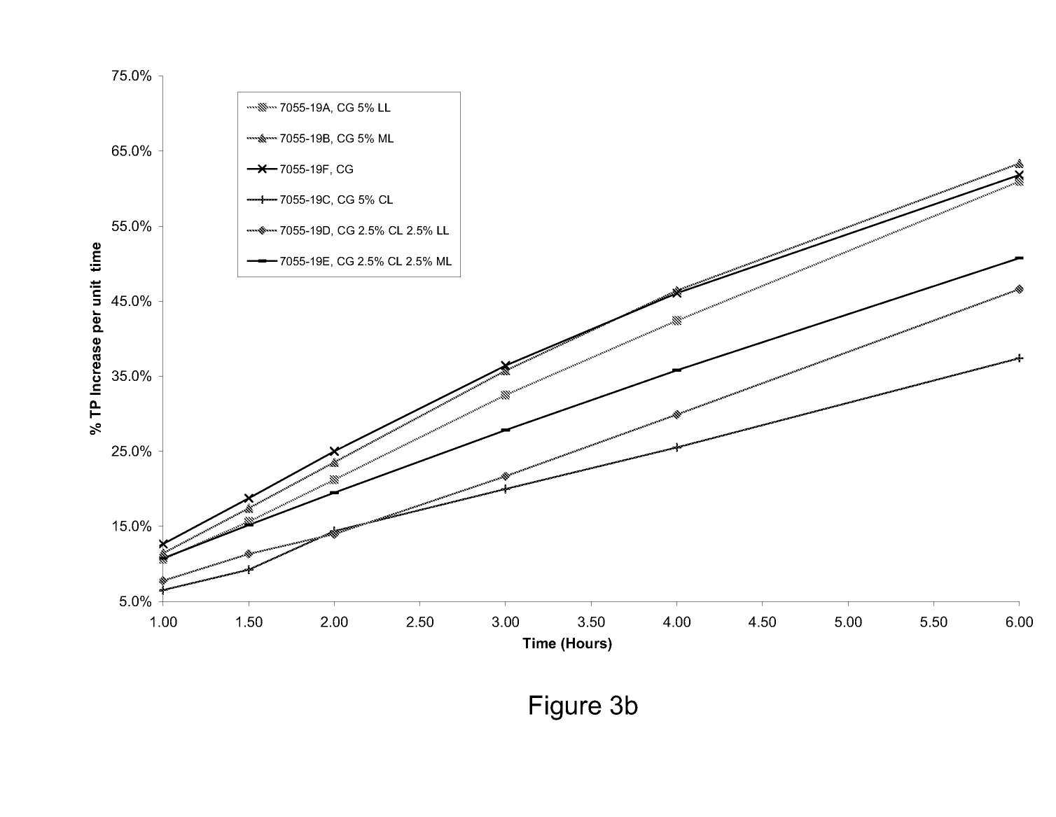

The above procedure can be used to produce a variety of formulations including a variety of ester mixtures. Some formulations produced by the above example are illustrated in Table 3 below. The % permeation of Ibuprofen through an Epiderm 200 Cultured Tissue for each of these formulations is illustrated in FIGS. 3a & 3b over a 6 hour time period (measured at pre-prescribed time points, i.e., 15, 30, 60, 90, 120, 360, 420 and 600 minutes post dose).

TABLE 3

| | | | | | 2.5% LL + | 2.5% ML + |
|---|---|---|---|---|---|---|
| | Control | 5% LL | 5% ML | 5% CL | 2.5% CL | 2.5% CL |
| | % w/w of Ingredients | | | | | |
| Ibuprofen | 5.00 | 5.00 | 5.00 | 5.00 | 5.00 | 5.00 |
| Caprilic Capric | 8.00 | 7.99 | 7.99 | 7.99 | 8.09 | 7.99 |
| Triglyceride | | | | | | |
| Octisalate | 5.00 | 5.00 | 5.00 | 5.00 | 5.00 | 5.00 |
| Lauryl lactate | 0.00 | 5.00 | 0.00 | 0.00 | 2.50 | 0.00 |
| Myristyl lactate | 0.00 | 0.00 | 5.00 | 0.00 | 0.00 | 2.50 |
| Cetyl lactate | 0.00 | 0.00 | 0.00 | 5.00 | 2.50 | 2.50 |
| DGME | 2.50 | 2.50 | 2.50 | 2.50 | 2.50 | 2.50 |
| Squalene | 2.00 | 2.00 | 2.00 | 2.00 | 2.00 | 2.00 |
| Sunflower Seed Oil | 2.00 | 2.10 | 2.10 | 2.00 | 2.00 | 2.10 |
| Phenyl Trimethicone | 5.50 | 5.00 | 5.00 | 5.09 | 5.00 | 5.00 |
| Dow Corning EL-8050 | 70.00 | 65.43 | 65.43 | 65.43 | 65.43 | 65.43 |
| Total Formulation % | 100.00 | 100.00 | 100.00 | 100.00 | 100.00 | 100.00 |

### Example 4

### Ibuprofen (5%) Hydroalcoholic Formulations

### General Preparation Procedure:

Water was weighted into a 50 mL beaker followed by addition of Carbapol® Ultrez 20. The resulting mixture was stirred until homogenous. Ibuprofen was then weighed into a 20 mL scintillation vial, followed by the addition of the appropriate amount of lauryl lactate (CHRYSTAPHYL®) (and/or other esters, e.g., myristyl lactate, cetyl lactate, stearyl lactate and/or lauryl mandelate) and EtOH. The vial was then capped and the mixture was sonicated for 30 min. to provide a clear, homogenous solution. This solution was then added to the Carbapol Ultrez 20 mixture portionwise with vigorous stirring to provide a slightly hazy, translucent gel. To the resulting mixture was added triisopropanolamine (TIA) in one portion followed by additional stirring until a clear, homogenous gel resulted.

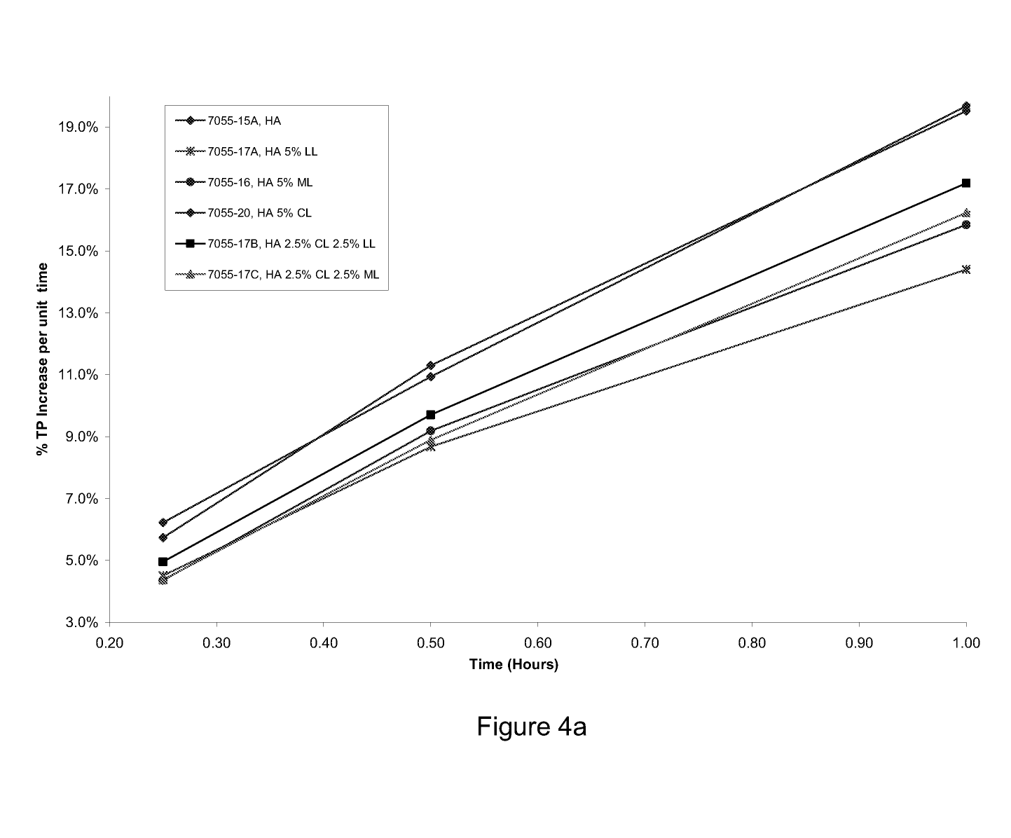

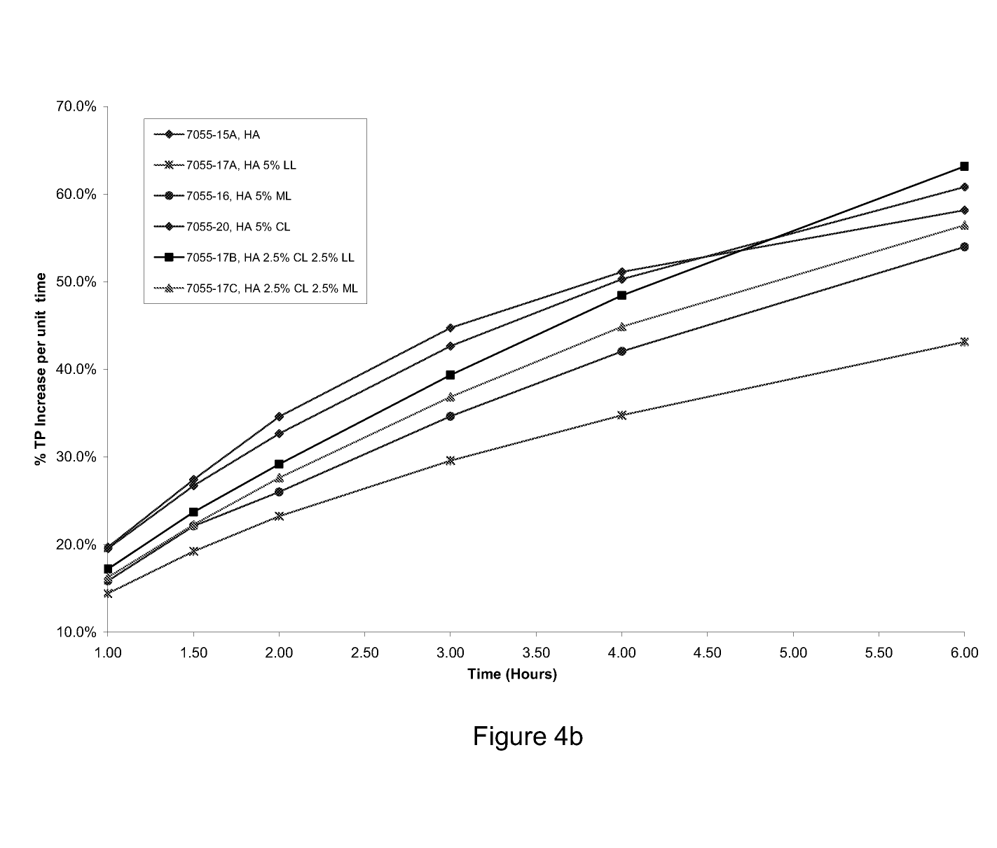

The above procedure can be used to produce a variety of hydroalcoholic formulations including a variety of ester mixtures. Some formulations produced by the above example are illustrated in Table 4 below. The % permeation of ibuprofen through an Epiderm 200 Cultured Tissue for each of these formulations is illustrated in FIGS. 4a & b over a 6 hour time period (measured at pre-prescribed time points, i.e., 15, 30, 60, 90, 120, 360, 420 and 600 minutes post dose).

TABLE 4

| | | | | | 2.5% LL + | 2.5% ML + |
|---|---|---|---|---|---|---|
| | Control | 5% LL | 5% ML | 5% CL | 2.5% CL | 2.5% CL |
| | % w/w of Ingredients | | | | | |
| water | 28.112 | 26.871 | 26.809 | 26.606 | 26.839 | 26.852 |
| Carbopol ® Ultrez 20 | 1.506 | 1.507 | 1.503 | 1.506 | 1.505 | 1.506 |
| Ibuprofen | 4.991 | 5.005 | 4.993 | 4.992 | 4.999 | 4.991 |
| Lauryl lactate | 0.00 | 4.991 | 0.00 | 0.00 | 2.496 | 0.00 |
| Myristyl lactate | 0.00 | 0.00 | 4.993 | 0.00 | 0.00 | 2.495 |
| Cetyl lactate | 0.00 | 0.00 | 0.00 | 4.992 | 2.496 | 2.495 |
| EtOH | 64.889 | 61.389 | 61.420 | 61.401 | 61.401 | 61.389 |
| Triisopropanolamine | 0.502 | 0.251 | 0.281 | 0.502 | 0.271 | 0.271 |
| (TIA) | | | | | | |
| Total Formulation % | 100.00 | 100.01 | 100.00 | 100.00 | 100.01 | 100.00 |

### Example 5

### Formulations Comprising Docosyl Lactate

### General Preparation Procedure

All components were weighed into a 40 mL clear glass vial, which was then placed in a hot oil bath (T_{bath}=90±10° C.). The mixture was stirred with a spatula as it was heated, eventually producing a homogeneous, translucent solution. Stirring was continued as the mixture cooled to RT to provide an opaque cream, which was then homogenized for 1-2 minutes (Ultra-Turrax 25 Basic equipped with a S25N-10G dispersing tool).

### A. Topical Cream Gel

| | % w/w | Trade Name/Supplier |
|---|---|---|
| 1. Cylcopentasiloxane | 40.00 | ST-Cyclomethicone |
| | | 5-NF/Dow Corning |
| 2. Docosyl lactate | 10.00 | Chemic Laboratories |
| 3. Myristyl lactate, 97+% | 10.00 | Chemic Laboratories |
| 4. Caprylic/capric | 10.00 | Labrafac Lipophile |
| triglyceride | | WL1349/Gattefosse |
| 5. Lauryl lactate, 98+% | 5.00 | Chrystaphyl ®/Chemic |
| | | Laboratories |
| 6. Octisalate | 5.00 | Spectrum |
| 7. Cyclopentasiloxane (and) | 5.00 | ST-Elastomer 10/Dow |
| dimethicone crosspolymer | | Corning |
| 8. Nylon-611/dimethicone | 5.00 | 28178 Gellant/Dow |
| copolymer (and) PPG-3 | | Corning |
| myristyl ether | | |
| 9. Coco-caprylate/caprate | 3.50 | Cetiol LC ®/Cognis |
| 10. L-Menthol, USP | 3.00 | Spectrum |
| 11. Aloe vera (aloe) oil | 2.00 | Concentrated Aloe |
| extract (and) coconut oil | | Corporation |
| 12. Vitamin E acetate, USP | 1.00 | Spectrum |
| 13. Vitamin A | 0.50 | Spectrum |
| palmitate, USP | | |

### B. Topical Cream Gel

| | % w/w | Trade Name/Supplier |
|---|---|---|
| 1. Cylcopentasiloxane | 40.00 | ST-Cyclomethicone |
| | | 5-NF/Dow Corning |
| 2. Docosyl lactate | 10.00 | Chemic Laboratories |
| 3. Myristyl lactate, 95+% | 10.00 | Chemic Laboratories |
| 4. Caprylic/capric | 10.00 | Labrafac Lipophile |
| triglyceride WL1349/ | | |
| Gattefosse | | |
| 5. Lauryl lactate, 97+% | 5.00 | Chrystaphyl ®/Chemic |
| | | Laboratories |
| 6. Octisalate | 5.00 | Spectrum |
| 7. Cyclopentasiloxane (and) | 5.00 | ST-Elastomer 10/ |
| dimethicone crosspolymer | | Dow Corning |
| 8. Nylon-611/dimethicone | 5.00 | 28178 Gellant/ |
| copolymer (and) PPG-3 | | Dow Corning |
| myristyl ether | | |
| 9. Coco-caprylate/caprate | 3.50 | Cetiol LC ®/Cognis |
| 10. C₁₂₋₁₅ alkyl benzoate | 3.00 | Finsolv TN ®/Innospec |
| 11. Menthyl lactate | 2.00 | Frescolat ® ML/ |
| | | Symrise 115 |
| 12. Vitamin E acetate, USP | 1.00 | Spectrum |
| 13. Vitamin A palmitate, | 0.50 | Spectrum |
| USP | | |

### C. Topical Cream Gel

| | % w/w | Trade Name/Supplier |
|---|---|---|
| 1. Cylcopentasiloxane | 25.00 | ST-Cyclomethicone |
| | | 5-NF/Dow Corning |
| 2. Docosyl lactate | 10.00 | Chemic Laboratories |
| 3. Myristyl lactate, 95+% | 10.00 | Chemic Laboratories |
| 4. Caprylic/capric | 10.00 | Labrafac Lipophile |
| triglyceride | | WL1349/Gattefosse |
| 5. Coco-caprylate/caprate | 10.00 | Cetiol ® LC/Cognis |
| 6. Oleyl erucate | 6.00 | Cettol ® J-600/Cognis |
| 7. Lauryl lactate, 97+% | 5.00 | Chrystaphyl ®/ |
| | | Chemic Laboratories |
| 8. Octisalate | 5.00 | Spectrum |
| 9. Nylon-611/dimethicone | 5.00 | 28178 Gellant/ |
| copolymer (and) PPG-3 | | Dow Corning |
| myristyl ether | | |
| 10. C₁₂₋₁₅ alkyl benzoate | 5.00 | Finsolv TN ®/Innospec |
| 11. Isostearyl neopentanoate | 5.00 | Crodamol |
| | | ISNP-LQ-(MH)/Croda |
| 12. Benzyl alcohol | 2.50 | Aldrich |
| 13. Vitamin E acetate, USP | 1.00 | Spectrum |
| 14. Vitamin A palmitate, | 0.50 | Spectrum |
| USP | | |

### D. Topical Cream Gel

| | % w/w | Trade Name/Supplier |
|---|---|---|
| 1. Dimethicone (and) | 25.00 | 2-1184 Fluid/Dow |
| trisiloxane | | Corning |
| 2. Docosyl lactate | 10.00 | Chemic Laboratories |
| 3. Myristyl lactate, 95+% | 10.00 | Chemic Laboratories |
| 4. Caprylic/capric | 10.00 | Labrafac Lipophile |
| triglyceride | | WL1349/Gattefosse |
| 5. Coco-caprylate/caprate | 10.00 | Cetiol ® LC/Cognis |
| 6. Oleyl erucate | 6.00 | Cetiol ® J-600/Cognis |
| 7. Lauryl lactate, 97+% | 5.00 | Chrystaphyl ®/ |
| | | Chemic Laboratories |
| 8. Octisalate | 5.00 | Spectrum |
| 9. Nylon-611/dimethicone | 5.00 | 28178 Gellant/Dow |
| copolymer (and) PPG-3 | | Corning |
| myristyl ether | | |
| 10. C₁₂₋₁₅ alkyl benzoate | 5.00 | Finsolv TN ®/Innospec |
| 11. Isostearyl | 5.00 | Crodamol |
| neopentanoate | | ISNP-LQ-(MH)/Croda |
| 12. Benzyl alcohol | 2.50 | Aldrich |
| 13. Vitamin E acetate, USP | 1.00 | Spectrum |
| 14. Vitamin A palmitate, | 0.50 | Spectrum |
| USP | | |

### E. Topical Cream Gel

| | % w/w | Trade Name/Supplier |
|---|---|---|
| 1. Hexamethyldisiloxane | 25.00 | Q7-9180 Silicone Fluid/ |
| (and) octamethyltrisiloxane | | Dow Corning |
| 2. Docosyl lactate | 10.00 | Chemic Laboratories |
| 3. Myristyl lactate, 95+% | 10.00 | Chemic Laboratories |
| 4. Caprylic/capric | 10.00 | Labrafac Lipophile |
| triglyceride | | WL1349/Gattefosse |
| 5. Coco-caprylate/caprate | 10.00 | Cetiol ® LC/Cognis |
| 6. Oleyl erucate | 6.00 | Cetiol ® J-600/Cognis |
| 7. Lauryl lactate, 97+% | 5.00 | Chrystaphyl ®/Chemic |
| | | Laboratories |
| 8. Octisalate | 5.00 | Spectrum |
| 9. Nylon-611/dimethicone | 5.00 | 28178 Gellant/ |
| copolymer (and) PPG-3 | | Dow Corning |
| myristyl ether | | |
| 10. C₁₂₋₁₅ alkyl benzoate | 5.00 | Finsolv TN ®/Innospec |
| 11. Isostearyl | 5.00 | Crodamol |
| neopentanoate | | ISNP-LQ-(MH)/Croda |
| 12. Benzyl alcohol | 2.50 | Aldrich |
| 13. Vitamin E acetate, USP | 1.00 | Spectrum |
| 14. Vitamin A palmitate, | 0.50 | Spectrum |
| USP | | |

### F. Topical Cream Gel

| | % w/w | Trade Name/Supplier |
|---|---|---|
| 1. Phenyl trimethicone | 25.00 | 556 Cosmetic Grade Fluid/ |
| | | Dow Corning |
| 2. Docosyl lactate | 10.00 | Chemic Laboratories |
| 3. Myristyl lactate, 95+% | 10.00 | Chemic Laboratories |
| 4. Caprylic/capric | 10.00 | Labrafac Lipophile |
| triglyceride | | WL1349/Gattefosse |
| 5. Coco-caprylate/caprate | 10.00 | Cetiol ® LC/Cognis |
| 6. Oleyl erucate | 6.00 | Cetiol ® J-600/Cognis |
| 7. Lauryl lactate, 97+% | 5.00 | Chrystaphyl ®/ |
| | | Chemic laboratories |
| 8. Octisalate | 5.00 | Spectrum |
| 9. Nylon-611/dimethicone | 5.00 | 28178 Gellant/ |
| copolymer (and) PPG-3 | | Dow Corning |
| myristyl ether | | |
| 10. C₁₂₋₁₅ alkyl benzoate | 5.00 | Finsolv TN ®/Innospec |
| 11. Isostearyl | 5.00 | Crodamol |
| neopentanoate | | ISNP-LQ-(MH)/Croda |
| 12. Benzyl alcohol | 2.50 | Aldrich |
| 13. Vitamin E acetate, USP | 1.00 | Spectrum |
| 14. Vitamin A palmitate, | 0.50 | Spectrum |
| USP | | |

### Example 6

### Flucinonide (0.05%) Anhydrous Formulations

Flucinonide is a potent topical anti-inflammatory that can be used to treat corticosteroid-responsive dermatoses, including eczema and psoriasis. A suitable topical formulation could be prepared by first mixing an appropriate amount of fluocinonide with caprylic triglyceride (Labrafac Lipophile WL 1349), octyl salicylate (Octisalate), phenyl trimethicone (556 Cosmetic Grade Fluid), squalene and sunflower seed oil. To this stock solution would then be added a mixture of lauryl lactate (CHRYSTAPHYL®) and/or other lactate esters, e.g., myristyl lactate, cetyl lactate, stearyl lactate, and/or lauryl mandelate. The resultant mixture could be gelled by the addition of isododecane dimethicone/bis-isobutyl PPG-20 crosspolymer (EL-8050 ID Silicone Organic Elastomer Blend), and thickened accordingly by the addition of silica silylate (VM-2270 Aerogel Fine Particles).

In the manner described herein, a variety of formulations could be produced. It is envisioned that by the judicious selection of lactate esters and their incorporation into the formulation, the transdermal penetration of fluocinonide and its corresponding residence time in the skin could be controlled so as to provide an optimal therapeutic effect.

The procedure described herein is not unique to fluocinonide, and other formulations can be conceived that would include hydrocortisone, fluocinolone or some other alternative topical corticosteroid.

### Example 7

### Celecoxib (2%) Hydroalcoholic Formulations

Celecoxib, an NSAID (non-steroidal anti-inflammatory drug), is a member of a class of compounds known as coxibs, which are selective inhibitors of cyclooxygenase type 2 (COX-2). The coxibs are used orally in the treatment of arthritis and other aberrant physiological processes accompanied by acute pain. Topical application of celecoxib may preclude serious adverse effects associated with oral use. A suitable topical formulation could be prepared by first dissolving celecoxib in ethanol, and then adding to the solution a mixture of lauryl lactate (CHRYSTAPHYL®) and/or other lactate esters, e.g., myristyl lactate, cetyl lactate, stearyl lactate, and/or lauryl mandelate. The resultant solution could be diluted with water, as required, and gelled by the addition of carbomer, followed by an appropriate base, e.g., trolamine.

In the manner described herein, a variety of formulations could be produced. It is envisioned that by the judicious selection of lactate esters and their incorporation into the formulation, the transdermal penetration of celecoxib and its corresponding residence time in the skin could be controlled so as to provide an optimal local or, if desired, systemic therapeutic effect.

The procedure described herein is not unique to celecoxib, and other formulations can be conceived that would include etoricoxib, rofecoxib, valdecoxib or some other member of this class.

### Example 8

### Tacrolimus Anhydrous Formulations

Tacrolimus is a potent immunomodulator that can be used topically (0.1%) to treat acute atopic dermatitis (eczema) and psoriasis. A topical formulation could be prepared by first making a solution of tacrolimus in ethanol and appropriate co-solvents and/or oils. Other compatible excipients that might include, but not be limited to: caprylic triglyceride (Labrafac Lipophile WL 1349), octyl salicylate (Octisalate), phenyl trimethicone (556 Cosmetic Grade Fluid), and squalene could also be added. To this solution would then be added a mixture of lauryl lactate (CHRYSTAPHYL®) and/or other lactate esters, e.g., myristyl lactate, cetyl lactate, stearyl lactate, and/or lauryl mandelate. The resultant mixture could be gelled by the addition of isododecane dimethicone/bis-isobutyl PPG-20 crosspolymer (EL-8050 ID Silicone Organic Elastomer Blend), and thickened accordingly by the addition of silica silylate (VM-2270 Aerogel Fine Particles).

In the manner described herein, a variety of formulations could be produced. It is envisioned that by the judicious selection of lactate esters and their incorporation into the formulation, the transdermal penetration of tacrolimus and its corresponding residence time in the skin could be controlled so as to provide an optimal therapeutic effect with, perhaps, a concomitant reduction in dose.

The procedure described herein is not unique to tacrolimus, and other formulations can be conceived that would include an alternative member of this therapeutic class like pimecrolimus.

1. A composition of esters, the composition comprising an α-hydroxy carboxylic acid ester and a second ester, wherein the α-hydroxy carboxylic acid ester and the second ester are not the same; and wherein less than 10% of the composition comprises fatty alcohols and/or alkyl lactyllactate and/or other related substances.

2. The composition of claim 1, wherein the second ester is a compound of formula (I):
second ester is a compound of formula (I): 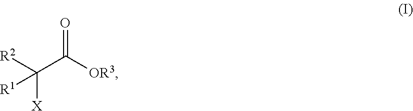
wherein
each R¹ is independently selected from the group consisting of: H, C₁-C₂₀ straight chained or branched alkyl, C₃-C₁₀ cycloalkyl, substituted C₁-C₂₀ alkyl, arylalkyl, aryl, substituted aryl and heteroaryl;
each R² is independently selected from the group consisting of: H and C₁-C₂₀ alkyl;
R³ is a C₁₂-C₄₀ alkyl; and
X is NR¹R¹ or OR¹;

3-5. (canceled)

6. The composition of claim 1, further comprising a third ester.

7. The composition of claim 6, wherein the α-hydroxy carboxylic acid ester, the second ester and the third ester are not the same.

8-11. (canceled)

12. The composition of claim 1, having less than 5% of the composition comprises fatty alcohols and/or alkyl lactyllactate and/or other related substances.

15-17. (canceled)

18. The composition of claim 1, wherein the composition is a pharmaceutical composition.

19. The composition of claim 1, wherein the composition is configured for topical administration or in the form of a patch.

20. The composition of claim 1, wherein the composition is in the form of an oil-in-water emulsion, a water-in-oil emulsion, a thickened aqueous gel, a hydrophilic gel or hydrophobic gel.

21. (canceled)

22. The composition of claim 1, consisting of an α-hydroxy carboxylic acid ester and a second ester, wherein the esters are not the same.

23. The composition of claim 1, further comprising a drug.

24-25. (canceled)

26. A composition of esters, the composition comprising an α-hydroxy carboxylic acid ester and a second ester, wherein the α-hydroxy carboxylic acid ester and the second ester are not the same; and wherein at least 90% of the composition is comprised of esters.

27. (canceled)

28. The composition of claim 26, wherein the second ester is a compound of formula (I):
second ester is a compound of formula (I): 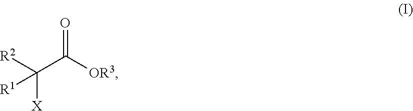
wherein
each R¹ is independently selected from the group consisting of: H, C₁-C₂₀ straight chained or branched alkyl, C₃-C₁₀ cycloalkyl, substituted C₁-C₂₀ alkyl, arylalkyl, aryl, substituted aryl and heteroaryl;
each R² is independently selected from the group consisting of: H and C₁-C₂₀ alkyl;
R³ is a C₁₂-C₄₀ alkyl; and
X is NR¹R¹ or OR¹.

29. The composition of claim 26, wherein the α-hydroxy carboxylic acid ester is a compound of formula (II): 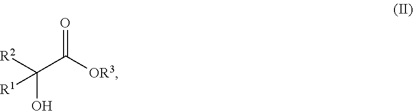
wherein R¹, R² and R³ are as defined in formula (I).

30-31. (canceled)

32. The composition of claim 26, wherein the composition is substantially free of fatty alcohols and/or alkyl lactyllactate and/or other related substances.

33. The composition of claim 26, further comprising a third ester.

34. The composition of claim 33, wherein the α-hydroxy carboxylic acid ester, the second ester and the third ester are not the same.

35-40. (canceled)

41. The composition of claim 26, wherein the composition is a pharmaceutical composition.

42. The composition of claim 26, wherein the composition is configured for topical administration or in the form of a patch.

43. The composition of claim 26, wherein the composition is in the form of an oil-in-water emulsion, a water-in-oil emulsion, a thickened aqueous gel or a hydrophilic gel.

44-45. (canceled)

46. The composition of claim 26, further comprising a drug.

47. (canceled)

48. A dosage form of a drug, the dosage form comprising a drug, and a composition comprising an α-hydroxy carboxylic acid ester and a second ester, wherein the two esters are not the same; and wherein less than 10% of the dosage form comprises fatty alcohols and/or alkyl lactyllactate and/or other related substances.

49-58. (canceled)

59. A method of administering a drug to a subject, the method comprising administering to the surface of a subject a composition comprising a drug, an α-hydroxy carboxylic acid ester and a second ester, wherein the two esters are not the same, and wherein less than 10% of the composition comprises comprises fatty alcohols and/or alkyl lactyllactate and/or other related substances.

60-67. (canceled)

68. A method of making a composition comprising a plurality of esters, the method comprising:
providing an α-hydroxy carboxylic acid ester with a purity of at least 90%;
providing a second ester with a purity of at least 90%; and
combining said first and second esters; wherein
the first and second esters are not the same.

69-80. (canceled)

81. A method of making a drug delivery device, comprising disposing composition comprising an α-hydroxy carboxylic acid ester and second ester on or in a said device; wherein
the first and second esters are not the same; and
wherein less than 10% of the device comprises fatty alcohols and/or alkyl lactyllactate and/or other related substances.

82-86. (canceled)

87. A method of providing a composition having a preselected value for a parameter rated to penetration, the method comprising:
selecting a value for the parameter;
selecting a mixture having of esters, the mixture having a relative amount of an α-hydroxy carboxylic acid ester and a second ester so as to provide for the selected value.

88-91. (canceled)

92. A device for sustained release of a drug comprising:
an optional adhesive element;
an optional drug storage element;
a drug;
an α-hydroxy carboxylic acid ester and a second ester, wherein the first and second esters are not the same, and wherein less than 10% of the device comprises fatty alcohols and/or alkyl lactyllactate and/or other related substances.

93-97. (canceled)
